(19) United States
(12) Patent Application Publication
(10) Pub. No.: US 2022/0160895 A1
(43) Pub. Date: May 26, 2022

(54) GENE THERAPY METHODS

(71) Applicant: Homology Medicines, Inc., Bedford, MA (US)

(72) Inventor: Janet Nancy ILES-SOMARATNE, Bedford, MA (US)

(21) Appl. No.: 17/453,685

(22) Filed: Nov. 5, 2021

Related U.S. Application Data
(60) Provisional application No. 63/110,257, filed on Nov. 5, 2020.

(51) Int. Cl. A61K 48/00; C07K 14/005; C12N 15/86

(52) U.S. Cl. CPC A61K 48/005; C07K 14/005; C12N 2830/001; C12N 2750/14122; C12N 2750/14143; C12N 15/86

(57) ABSTRACT

Provided are methods for reducing the risk of occurrence and/or the severity of pathogenic diseases (e.g., diseases associate with varicella zoster virus reactivation, influenza infection, and/or S. pneumoniae infection) in a subject that is receiving a gene therapy and an accompanying immunosuppressant regimen.

### RELATED APPLICATIONS

This application claims priority to U.S. Provisional Patent Application Ser. No. 63/110,257, filed Nov. 5, 2020, the entire disclosure of which is hereby incorporated herein by reference.

### REFERENCE TO SEQUENCE LISTING SUBMITTED ELECTRONICALLY

The content of the electronically submitted sequence listing in ASCII text file (Name: “404217-HMW-046US_187031_ST25.txt”; Size: 137,012 bytes; and Date of Creation: Nov. 5, 2021) is incorporated herein by reference in its entirety.

### BACKGROUND

Gene therapy holds great promise for the treatment of genetic diseases. Recombinant viral vectors (e.g., lentiviral, adenoviral, and an adeno-associated viral vectors) have been demonstrated to be useful for delivering functional copies of genes into the cells of subjects, to correct genetic defects and treat the diseases caused by those defects. Viral vector-based gene therapies are generally administered to a subject in conjunction with an immunosuppressant drug regime, to reduce the risk that the subject will mount a deleterious immune response against the viral vector. However, the immunosuppressant drug regime increases the risk that subject will develop a pathogenic disease that may impact the efficacy of the gene therapy.

Accordingly, there is a need for improved gene therapy methods that can reduce the risk of pathogenic disease in subjects receiving a gene therapy and an immunosuppressant regime.

### SUMMARY

The instant disclosure provides methods for reducing the risk of occurrence and/or the severity of pathogenic diseases (e.g., diseases associated with varicella zoster virus reactivation, influenza infection, and/or S. pneumoniae infection) in a subject that is receiving a gene therapy and an accompanying immunosuppressant regimen. Such methods are particularly advantageous in that they reduce the risk of the immunosuppressant regimen being discontinued due to pathogenic infection in the subject, and, in turn, reduce the risk that the gene therapy will be ineffective.

Accordingly, in one aspect, the instant disclosure provides a method for reducing the risk of occurrence and/or the severity of a pathogenic disease in a subject receiving a gene therapy and an immunosuppressant regimen, the method comprising: administering a prophylactic vaccine to a subject that will receive the gene therapy and the immunosuppressant regimen.

In certain embodiments, the pathogenic disease is herpes zoster and the method comprises: administering a herpes zoster vaccine to a subject that will receive the gene therapy and the immunosuppressant regimen, wherein an initial dose of the herpes zoster vaccine is administered to the subject prior to administration of the immunosuppressant regimen.

In certain embodiments, the vaccine comprises a polysaccharide, a polypeptide, or a nucleic acid. In certain embodiments, the vaccine is a subunit vaccine. In certain embodiments, the vaccine comprises a varicella zoster virus glycoprotein E antigen. In certain embodiments, the vaccine comprises a recombinant varicella zoster virus glycoprotein E antigen. In certain embodiments, the vaccine comprises recombinant varicella zoster virus glycoprotein E antigen, monophosphoryl lipid A, and QS-21.

In certain embodiments, the initial dose of the vaccine is administered to the subject at least about 6 weeks prior to commencement of the immunosuppressant regimen.

In certain embodiments, at least one subsequent dose of the vaccine is administered to the subject after administration of the initial dose. In certain embodiments, the at least one subsequent dose of the vaccine is administered to the subject at least about 2 weeks prior to commencement of the immunosuppressant regimen.

In certain embodiments, the gene therapy is administered to the subject at least 1 day after commencement of the immunosuppressant regimen.

In certain embodiments, the pathogenic disease is an S. pneumoniae related disease or disorder and the method comprises: administering an S. pneumoniae vaccine to a subject that will receive the gene therapy and the immunosuppressant regimen, wherein an initial dose of the vaccine is administered to the subject prior to administration of the immunosuppressant regimen.

In certain embodiments, the S. pneumoniae related disease or disorder is selected from the group consisting of pneumonia, meningitis, sepsis, and any combination thereof.

In certain embodiments, the vaccine comprises a polysaccharide, a polypeptide, or a nucleic acid. In certain embodiments, the vaccine is selected from the group consisting of: a live-attenuated vaccine; an inactivated vaccine; a subunit vaccine; a toxoid vaccine; and a nucleic acid vaccine. In certain embodiments, the vaccine is a subunit vaccine selected from the group consisting of a polysaccharide vaccine, a conjugate vaccine, a toxoid vaccine, and a recombinant protein vaccine.

In certain embodiments, the vaccine is a conjugate vaccine. In certain embodiments, the conjugate vaccine comprises purified capsular polysaccharides of one or more of serotypes 1, 3, 4, 5, 6A, 6B, 7F, 9V, 14, 18C, 19A, 19F, and 23F of S. pneumoniae conjugated to CRM197. In certain embodiments, the conjugate vaccine comprises purified capsular polysaccharides of serotypes 1, 3, 4, 5, 6A, 6B, 7F, 9V, 14, 18C, 19A, 19F, and 23F of S. pneumoniae conjugated to CRM197 (PCV13).

In certain embodiments, the vaccine is a polysaccharide vaccine. In certain embodiments, the polysaccharide vaccine comprises purified capsular polysaccharides of one or more of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae. In certain embodiments, the polysaccharide vaccine comprises purified capsular polysaccharides of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae (PPSV23).

In certain embodiments, the initial dose of the vaccine comprises PCV13.

In certain embodiments, the initial dose of the vaccine is administered to the subject at least about 10 weeks prior to commencement of the immunosuppressant regimen.

In certain embodiments, at least one subsequent dose of the vaccine is administered to the subject at least about 8 weeks after administration of the initial dose. In certain embodiments, the at least one subsequent dose of the vaccine comprises PPSV23. In certain embodiments, the at least one subsequent dose of the vaccine is administered to the subject at least about 2 weeks prior to commencement of the immunosuppressant regimen.

In certain embodiments, the gene therapy is administered to the subject at least 1 day after commencement of the immunosuppressant regimen.

In certain embodiments, the pathogenic disease is influenza, and the method comprises administering an influenza vaccine to a subject that will receive or has received the gene therapy and the immunosuppressant regimen.

In certain embodiments, the vaccine comprises a polysaccharide, a polypeptide, or a nucleic acid. In certain embodiments, the vaccine is selected from the group consisting of: a live-attenuated vaccine; an inactivated vaccine; a subunit vaccine; a toxoid vaccine; and a nucleic acid vaccine. In certain embodiments, the vaccine is a subunit vaccine selected from the group consisting of a polysaccharide vaccine, a conjugate vaccine, a toxoid vaccine, and a recombinant protein vaccine. In certain embodiments, the vaccine is a nucleic acid vaccine selected from the group consisting of a DNA-based vaccine, an RNA-based vaccine, and a recombinant vector vaccine.

In certain embodiments, the vaccine is administered to the subject prior to commencement of the immunosuppressant regimen. In certain embodiments, the vaccine is administered to the subject at least about 2 weeks prior to commencement of the immunosuppressant regimen. In certain embodiments, the vaccine is administered to the subject after commencement of the immunosuppressant regimen.

In certain embodiments, the gene therapy is administered to the subject at least 1 day after commencement of the immunosuppressant regimen.

In certain embodiments, the immunosuppressant regimen comprises administration of a glucocorticoid. In certain embodiments, the glucocorticoid is selected from the group consisting of hydrocortisone, cortisone acetate, prednisone, prednisolone, methylprednisolone (methylprednisone), triamcinolone, dexamethasone, and betamethasone.

In certain embodiments, the gene therapy comprises a recombinant viral vector. In certain embodiments, the recombinant viral vector comprises a transgene. In certain embodiments, the transgene encodes a polypeptide.

In certain embodiments:

the polypeptide is selected from the group consisting of β-globin, hemoglobin, tissue plasminogen activator, and coagulation factors; colony stimulating factors (CSF);
the polypeptide is an interleukin, optionally wherein the interleukin is selected from the group consisting of IL-1, IL-2, IL-3, IL-4, IL-5, IL-6, IL-7, IL-8, and IL-9; the polypeptide is a growth factor, optionally wherein the growth factor is selected from the group consisting of a keratinocyte growth factor (KGF), stem cell factor (SCF), fibroblast growth factor (FGF), basic FGF, acidic FGF, hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), a neurotrophin, platelet-derived growth factor (PDGF), thrombopoietin (TPO), transforming growth factor alpha (TGF-a), and transforming growth factor beta (TGF-β);
the polypeptide is a soluble receptor, optionally wherein the soluble receptor is selected from the group consisting of a soluble TNF-α receptor, a soluble interleukin receptor, a soluble γ/Δ T cell receptor, and ligand-binding fragments of a soluble receptor;
the polypeptide is an enzyme, optionally wherein the enzyme is selected from the group consisting of a-glucosidase, imiglucerase, β-glucocerebrosidase, and alglucerase;
the polypeptide is an enzyme activator, optionally wherein the enzyme activator is tissue plasminogen activator;
the polypeptide is a chemokine, optionally wherein the chemokine is selected from the group consisting of IP-10, monokine induced by interferon-gamma (Mig), Groα/IL-8, RANTES, MIP-1a, MIP-1β, MCP-1, and PF-4;
the polypeptide is an angiogenic agent, optionally wherein the angiogenic agent is VEGF, VEGF121, VEGF165, VEGF-C, VEGF-2, glioma-derived growth factor, angiogenin, and angiogenin-2;
the polypeptide is an anti-angiogenic agent, optionally wherein the anti-angiogenic agent is selected from the group consisting of a soluble VEGF receptor;
the polypeptide is a protein vaccine;
the polypeptide is a neuroactive peptide, optionally wherein the neuroactive peptide is selected from the group consisting of a nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, a glucagon, vasopressin, angiotensin II, thyrotropin-releasing hormone, vasoactive intestinal peptide, and a sleep peptide;
the polypeptide is selected from the group consisting of a thrombolytic agent, atrial natriuretic peptide, relaxin, glial fibrillary acidic protein, follicle stimulating hormone (FSH), human alpha-1 antitrypsin, leukemia inhibitory factor (LIF), a tissue factor, a macrophage activating factor, a tumor necrosis factor (TNF), neutrophil chemotactic factor (NCF), a tissue inhibitor of a metalloproteinase, vasoactive intestinal peptide, angiogenin, angiotrophin, fibrin, hirudin, an IL-1 receptor antagonist, ciliary neurotrophic factor (CNTF), brain-derived neurotrophic factor (BDNF), neurotrophin 3, neurotrophin 4/5, glial cell derived neurotrophic factor (GDNF), aromatic amino acid decarboxylase (AADC), Factor VIII, Factor IX, Factor X, dystrophin, mini-dystrophin, lysosomal acid lipase, and phenylalanine hydroxylase (PAH);
the polypeptide is a glycogen storage disease-related enzyme, optionally wherein the glycogen storage disease-related enzyme is selected from the group consisting of glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, and glycogen synthase;
the polypeptide is a lysosomal enzyme, optionally wherein the lysosomal enzyme is selected from the group consisting of iduronate-2-sulfatase (I2S), and arylsulfatase A;
the polypeptide is a mitochondrial protein, optionally wherein the mitochondrial protein is frataxin (FXN);
the polypeptide is a protein that may be defective in one or more lysosomal storage diseases, wherein the protein is selected from the group consisting of α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, 0-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN6, CLN8, LYST, MYOV, RAB27A, melanophilin, and AP3 β-subunit;
the polypeptide is an antibody or fragment thereof, optionally wherein the antibody is selected from the group consisting of muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab;
the polypeptide is a nuclease, optionally wherein the nuclease is selected from the group consisting of a zinc finger nuclease (ZFN), a transcription activator-like effector nuclease (TALEN), a homing endonuclease, and a meganuclease;
the polypeptide is an RNA-guided nuclease, wherein the RNA-guided nuclease is selected from the group consisting of a Cas3, Cas8a, Cas5, Cas8b, Cas8c, Cas10d, Cse1, Cse2, Csy1, Csy2, Csy3, GSU0054, Cas10, Csm2, Cmr5, Csx10, Csx11, Csx10, Csf1, Cas9, Csn2, Cas4, Cpf1, C2c1, C2c3, Cas13a, Cas13b, Cas13c, and Cas12a/Cpf1; or
the polypeptide is a reporter, optionally wherein the reporter is selected from the group consisting of β-lactamase, β-galactosidase (LacZ), alkaline phosphatase, thymidine kinase, green fluorescent protein (GFP), red fluorescent protein (RFP), chloramphenicol acetyltransferase (CAT), luciferase, CD2, CD4, CD8, the influenza hemagglutinin protein, and Myc.

In certain embodiments, the transgene encodes an miRNA, shRNA, siRNA, antisense RNA, gRNA, antagomir, miRNA sponge, RNA aptazyme, RNA aptamer, lncRNA, ribozyme or mRNA.

In certain embodiments, the recombinant viral vector is selected from the group consisting of a retroviral vector, a lentiviral vector, an adenoviral vector, and an adeno-associated virus vector. In certain embodiments, the recombinant viral vector is an adeno-associated virus (AAV) vector.

In certain embodiments, the adeno-associated virus (AAV) vector comprises:

(a) an AAV capsid comprising an AAV capsid protein; and
(b) a recombinant AAV genome.

In certain embodiments, the AAV capsid protein is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PUP.S.

In certain embodiments, the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17. In certain embodiments: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments:

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17. In certain embodiments: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments:

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the AAV capsid protein comprises the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17. In certain embodiments: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments:

(a) the amino acid in the capsid protein corresponding to amino acid 2 of SEQ ID NO: 16 is T, and the amino acid in the capsid protein corresponding to amino acid 312 of SEQ ID NO: 16 is Q;
(b) the amino acid in the capsid protein corresponding to amino acid 65 of SEQ ID NO: 16 is I, and the amino acid in the capsid protein corresponding to amino acid 626 of SEQ ID NO: 16 is Y;
(c) the amino acid in the capsid protein corresponding to amino acid 77 of SEQ ID NO: 16 is R, and the amino acid in the capsid protein corresponding to amino acid 690 of SEQ ID NO: 16 is K;
(d) the amino acid in the capsid protein corresponding to amino acid 119 of SEQ ID NO: 16 is L, and the amino acid in the capsid protein corresponding to amino acid 468 of SEQ ID NO: 16 is S;
(e) the amino acid in the capsid protein corresponding to amino acid 626 of SEQ ID NO: 16 is G, and the amino acid in the capsid protein corresponding to amino acid 718 of SEQ ID NO: 16 is G;
(f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(g) the amino acid in the capsid protein corresponding to amino acid 505 of SEQ ID NO: 16 is R, and the amino acid in the capsid protein corresponding to amino acid 687 of SEQ ID NO: 16 is R;
(h) the amino acid in the capsid protein corresponding to amino acid 346 of SEQ ID NO: 16 is A, and the amino acid in the capsid protein corresponding to amino acid 505 of SEQ ID NO: 16 is R; or
(i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the recombinant AAV genome comprises from 5′ to 3′: an ApoE-HCR-hAAT promoter; a composite globin/AIAT intron; a codon optimized human phenylalanine hydroxylase coding sequence; and a bovine growth hormone polyadenylation signal. In certain embodiments, the recombinant AAV genome comprises the nucleic acid sequence of SEQ ID NO: 23. In certain embodiments, the AAV capsid is an AAV5 capsid.

In another aspect, the instant disclosure provides a method of treating a subject having a disease or disorder, the method comprising administering to the subject a gene therapy, wherein the subject has received a first prophylactic vaccine, and wherein the prophylactic vaccine reduces the risk of occurrence and/or the severity of a first pathogenic disease in the subject.

In certain embodiments, the first prophylactic vaccine is a herpes zoster vaccine.

In certain embodiments, the subject has received a second prophylactic vaccine, and wherein the second prophylactic vaccine is an S. pneumoniae vaccine.

In certain embodiments, the subject has received a third prophylactic vaccine, and wherein the third prophylactic vaccine is an influenza vaccine.

In certain embodiments, the disease or disorder is phenylketonuria (PKU).

In certain embodiments, the gene therapy is mediated by a recombinant viral vector. In certain embodiments, the recombinant viral vector comprises a transgene encoding phenylalanine hydroxylase (PAH). In certain embodiments, the recombinant viral vector is an adeno-associated virus (AAV) vector.

In certain embodiments, the adeno-associated virus (AAV) vector comprises:

(a) an AAV capsid comprising an AAV capsid protein; and
(b) a recombinant AAV (rAAV) genome comprising a transgene encoding a phenylalanine hydroxylase (PAH).

In certain embodiments, the transgene comprises the nucleotide sequence set forth in SEQ ID NO: 28. In certain embodiments, the rAAV genome further comprises a transcriptional regulatory element operably linked to the PAH coding sequence. In certain embodiments, the transcriptional regulatory element is capable of mediating transcription in a hepatocyte, a renal cell, or a cell in the brain, pituitary gland, adrenal gland, pancreas, urinary bladder, gallbladder, colon, small intestine, or breast. In certain embodiments, the transcriptional regulatory element comprises a human hepatic control region 1 (HCR1) comprising the nucleotide sequence set forth in SEQ ID NO: 24. In certain embodiments, the transcriptional regulatory element comprises a human al-antitrypsin (hAAT) promoter comprising the nucleotide sequence set forth in SEQ ID NO: 25. In certain embodiments, the transcriptional regulatory element comprises an SV40 intron comprising the nucleotide sequence set forth in SEQ ID NO: 26. In certain embodiments, the transcriptional regulatory element comprises the nucleotide sequence set forth in SEQ ID NO: 27.

In certain embodiments, the rAAV genome further comprises an SV40 polyadenylation sequence 3′ to the PAH coding sequence, wherein the SV40 polyadenylation sequence comprises the nucleotide sequence set forth in SEQ ID NO: 29.

In certain embodiments, the rAAV genome comprises the nucleotide sequence set forth in SEQ ID NO: 32.

In certain embodiments, the rAAV genome further comprises a 5′ inverted terminal repeat (5′ ITR) nucleotide sequence 5′ of the genome, and a 3′ inverted terminal repeat (3′ ITR) nucleotide sequence 3′ of the genome. In certain embodiments, the 5′ ITR nucleotide sequence has at least 95% sequence identity to SEQ ID NO: 30, and the 3′ ITR nucleotide sequence has at least 95% sequence identity to SEQ ID NO: 31.

In certain embodiments, the rAAV genome comprises the nucleotide sequence set forth in SEQ ID NO: 33.

In certain embodiments, the AAV capsid comprises:

a capsid protein comprising an amino acid sequence having at least 95% sequence identity with the amino acid sequence of amino acids 203-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
a capsid protein comprising an amino acid sequence having at least 95% sequence identity with the amino acid sequence of amino acids 138-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R; and/or
a capsid protein comprising an amino acid sequence having at least 95% sequence identity with the amino acid sequence of amino acids 1-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R.

In certain embodiments, the AAV capsid comprises:

a capsid protein comprising an amino acid sequence having at least 99% sequence identity with the amino acid sequence of amino acids 203-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
a capsid protein comprising an amino acid sequence having at least 99% sequence identity with the amino acid sequence of amino acids 138-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R; and/or
a capsid protein comprising an amino acid sequence having at least 99% sequence identity with the amino acid sequence of amino acids 1-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R.

In certain embodiments, the AAV capsid comprises a capsid protein comprising the amino acid sequence of amino acids 203-736 of SEQ ID NO: 16, a capsid protein comprising the amino acid sequence of amino acids 138-736 of SEQ ID NO: 16, and/or a capsid protein comprising the amino acid sequence of amino acids 1-736 of SEQ ID NO: 16.

In certain embodiments, the amino acid sequence of the capsid protein consists of the amino acid sequence of amino acids 203-736 of SEQ ID NO: 16, the amino acid sequence of the capsid protein consists of the amino acid sequence of amino acids 138-736 of SEQ ID NO: 16, and/or the amino acid sequence of the capsid protein consists of the amino acid sequence of amino acids 1-736 of SEQ ID NO: 16.

In certain embodiments, the transgene comprises the nucleotide sequence set forth in SEQ ID NO: 22. In certain embodiments, the transcriptional regulatory element comprises an ApoE-HCR element, optionally comprising the nucleotide sequence set forth in SEQ ID NO: 19. In certain embodiments, the transcriptional regulatory element comprises a human al-antitrypsin (hAAT) promoter comprising the nucleotide sequence set forth in SEQ ID NO: 20. In certain embodiments, the transcriptional regulatory element comprises a composite globin/AIAT intron, optionally comprising the nucleotide sequence set forth in SEQ ID NO: 18. In certain embodiments, the transcriptional regulatory element comprises the nucleotide sequence set forth in SEQ ID NO: 21. In certain embodiments, the rAAV genome further comprises a bovine growth hormone polyadenylation sequence. In certain embodiments, the rAAV genome further comprises an AAV2 5′ inverted terminal repeat (5′ ITR) nucleotide sequence 5′ of the genome, and an AAV2 3′ inverted terminal repeat (3′ ITR) nucleotide sequence 3′ of the genome. In certain embodiments, the rAAV genome comprises the nucleotide sequence set forth in SEQ ID NO: 23. In certain embodiments, the AAV capsid is an AAV5 capsid.

### BRIEF DESCRIPTION OF DRAWINGS

FIG. 1 is a vector map of the pHMI-hPAH-TC-025 vector.

FIGS. 2A-2B are graphs showing the levels of phenylalanine over time in the serum of male (FIG. 2A) or female (FIG. 2B) mice administered the indicated doses of an rAAV comprising the pHMI-hPAH-TC-025 vector.

FIGS. 3A-3D are graphs showing the levels of phenylalanine (FIGS. 3A and 3C) or tyrosine (FIGS. 3B and 3D) in the serum of male (FIGS. 3A and 3B) or female (FIGS. 3C and 3D) mice administered with the indicated doses of an rAAV comprising the pHMI-hPAH-TC-025 vector.

FIG. 4 is a schematic showing the design of an open-label, randomized, concurrently-controlled, dose escalation study of a single, ascending dose of an rAAV comprising the pHMI-hPAH-TC-025 vector packaged in AAVHSC15 capsid, in adult subjects with PAH deficiency.

FIG. 5 is a schematic showing the design of an open-label, randomized, concurrently-controlled, dose escalation study of a single, ascending dose of an rAAV comprising the pHMI-hPAH-TC-025 vector packaged in AAVHSC15 capsid, in adult subjects with PAH deficiency.

### DETAILED DESCRIPTION

The instant disclosure provides methods for reducing the risk of occurrence and/or the severity of pathogenic diseases (e.g., diseases associate with varicella zoster virus reactivation, influenza infection, and/or S. pneumoniae infection) in a subject that is receiving a gene therapy and an accompanying immunosuppressant regimen. Such methods are particularly advantageous in that they reduce the risk of the immunosuppressant regimen being discontinued due to pathogenic infection in the subject, and, in turn, reduce the risk that the gene therapy will be ineffective.

### I. Definitions

As used herein, the term “AAV” is a standard abbreviation for adeno-associated virus.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein.

As used herein, the term “rep gene” refers to the nucleic acid sequences that encode the non-structural proteins (e.g., rep78, rep68, rep52 and rep40) required for the replication and production of an AAV.

As used herein, the term “transfer genome” refers to a recombinant AAV genome comprising a coding sequence operably linked to an exogenous transcriptional regulatory element that mediates expression of the coding sequence when the transfer genome is introduced into a cell. In certain embodiments, the transfer genome does not integrate in the chromosomal DNA of the cell. The skilled artisan will appreciate that the portion of a transfer genome comprising the transcriptional regulatory element operably linked to a transgene can be in the sense or antisense orientation relative to direction of transcription of the transgene.

As used herein, the term “gene therapy” refers to the delivery of a transgene into a cell in order to correct a genetic disorder. In certain embodiments, the gene therapy is mediated by a recombinant viral vector, e.g., a retroviral vector, a lentiviral vector, an adenoviral vector, and an adeno-associated virus vector. In general, a recombinant viral vector comprises a transgene, optionally wherein the transgene is operably linked to a transcriptional regulatory element.

As used herein, the term “liver-directed gene therapy” refers to a gene therapy that is capable of transducing a liver cell (e.g., a hepatocyte) thereby resulting in expression of the transgene in the liver cell.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches, and does not consider the degree of similarity of amino acids to one another. Note that only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may either be wild-type or codon-altered (e.g., codon optimized).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “polyadenylation sequence” refers to a DNA sequence that when transcribed into RNA constitutes a polyadenylation signal sequence. The polyadenylation sequence can be native (e.g., from the PAH gene) or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a transgene. In certain embodiments, an exogenous polyadenylation sequence is a polyadenylation sequence of a gene different from the transgene, but within the same species (e.g., human). In certain embodiments, an exogenous polyadenylation sequence is a polyadenylation sequence of a different species (e.g., a virus).

As used herein, the term “about” or “approximately” when referring to a measurable value, such as a dosage, encompasses variations of +20% or ±10%, +5%, +1%, or ±0.1% of a given value or range, as are appropriate to perform the methods disclosed herein.

As used herein in the context of the result of a liver function test (e.g., the level of a liver transaminase in the blood of a subject), the term “normal range,” refers to a reference range expected for a healthy subject (i.e., a non-pathophysiological reference range). It is appreciated by those of skill in the art that a reference range varies between laboratory testing sites. As such, when determining whether a test value is within a normal range, the reference range supplied by the laboratory testing site that obtained the test value should be used. Further, it is known in the art that a reference range for a certain liver function test may be different for male and female sexes. Common liver function tests include determining the level of alanine aminotransferase (ALT), aspartate aminotransferase (AST), alkaline phosphate (ALP), gamma-glutamyltransferase (GGT), bilirubin, and albumin. Liver function tests also include determining the prothrombin time (PT), which is a test that measures how long it takes blood to clot. In certain embodiments, the reference range for ALT is from 0 to about 45 IU/L, from about 3 to about 30 U/L, from about 1 to about 45 U/L, from about 17 to about 63 U/L, from about 14 to about 54 U/L. In certain embodiments, the reference range for AST is from 0 to about 35 IU/L, from about 2 to about 40 U/L, from about 1 to about 35 U/L, from about 18 to about 57 U/L, from about 5 to about 34 U/L, from about 15 to about 41 U/L. In certain embodiments, the reference range for ALP is from about 30 to about 120 IU/L, from about 38 to about 126 U/L, from about 69 to about 318 U/L, from about 53 to about 212 U/L, from about 34 to about 104 U/L. In certain embodiments, the reference range for direct bilirubin is from about 2 to about 17 μmol/L, from about 0 to about 0.4 mg/dL, from about 0 to about 0.8 mg/dL, from about 0 to about 0.3 mg/dL, from about 0 to about 0.2 mg/dL. In certain embodiments, the reference range for total bilirubin is from about 0.3 to about 1.2 mg/dL, from about 0.1 to about 1.2 mg/dL, from about 0.2 to about 1.2 mg/dL. In certain embodiments, the reference range for prothrombin time is from about 10.9 to about 12.5 seconds. In certain embodiments, the reference range for albumin is from about 40 to about 60 g/L.

As used herein in the context of the result of a liver function test (e.g., the level of a liver transaminase in the blood of a subject), “baseline value,” refers to a result of a liver function test that was obtained prior to the administration of a treatment described herein (e.g., administration of a gene therapy to the subject). In certain embodiments, the baseline value for a liver function test is the result of the liver function test obtained from the subject prior to the administration of the gene therapy (e.g., a liver directed gene therapy). For example, the baseline value for ALT and/or AST is the value of ALT and/or AST obtained from the subject prior to the administration of a gene therapy.

In certain embodiments, the result of a liver function test may be reported as a multiple of a certain reference value. For example, the result of a liver function test may be reported as a multiple of an upper limit of normal. As used herein, the term “upper limit of normal” or “ULN,” refers to the upper value of a reference range. For example, the ULN for ALT is the upper value of the reference range for ALT. In certain embodiments, the ULN for ALT is about 45 IU/L, about 30 U/L, about 45 U/L, about 63 U/L, about 54 U/L. In certain embodiments, the ULN for ALT is from about 30 U/L to about 63 U/L. In certain embodiments, the ULN for AST is about 35 IU/L, about 40 U/L, about 57 U/L, about 34 U/L, about 41 U/L. In certain embodiments, the ULN for AST is from about 34 U/L to about 57 U/L. In certain embodiments, the ULN for ALP is about 120 IU/L, about 126 U/L, about 318 U/L, about 212 U/L, about 104 U/L. In certain embodiments, the ULN for ALP is from about 104 U/L to about 318 U/L. In certain embodiments, the ULN for direct bilirubin is about 17 μmol/L, about 0.4 mg/dL, about 0.8 mg/dL, about 0.3 mg/dL, about 0.2 mg/dL. In certain embodiments, the ULN for direct bilirubin is from about 0.2 mg/dL to about 0.8 mg/dL. In certain embodiments, the ULN for total bilirubin is about 1.2 mg/dL. In certain embodiments, the ULN for prothrombin time is about 12.5 seconds. In certain embodiments, the ULN for albumin is about 60 g/L. As such, the result of a liver function test may be reported as, e.g., at least about 1.5 times the ULN, at least about 2 times the ULN, at least about 2.5 times the ULN, at least about 3 times the ULN, at least about 4 times the ULN, at least 5 times the ULN, at least 20 times the ULN, and the like.

An event such as elevated ALT can be described by a certain Grade. As used herein, the term “Grade” when used in the context of an event, refers to the Grade designation as provided by Common Terminology Criteria for Adverse Events (CTCAE). For example, the level of ALT elevation can be described as Grade 1 (greater than about 1 to about 3 times ULN if baseline was normal; greater than about 1.5 to about 3 times baseline if baseline was abnormal), Grade 2 (greater than about 3 to about 5 times ULN if baseline was normal; greater than about 3 to about 5 times baseline if baseline was abnormal), Grade 3 (greater than about 5 to about 20 times ULN if baseline was normal; greater than about 5 to about 20 times baseline if baseline was abnormal), and Grade 4 (greater than about 20 times ULN if baseline was normal; greater than about 20 times baseline if baseline was abnormal).

As used herein, the term “subunit vaccine” refers to a vaccine comprising a polypeptide or a glycoprotein component of a pathogen.

As used herein, the term “polysaccharide vaccine” refers to a vaccine comprising polysaccharide components of a pathogen.

As used herein, the term “conjugate vaccine” refers to a vaccine comprising components of a pathogen linked to a strong antigenic carrier, e.g., a mutant form of a diphtheria toxin.

As used herein, the term “CRM197” refers to the mutant form of diphtheria toxin described in Shinefield, H. R. (2010). Vaccine. 28(27): 4335-4339.

### II. Prophylactic Vaccines

Applicants have determined that there is an increased risk of occurrence of a pathogenic disease in a subject receiving a gene therapy and an immunosuppressant regimen. The development of pathogenic disease in such a subject is highly undesirable because it would necessitate discontinuation of the immunosuppressant regimen, which, in turn, may lead to a failure of the gene therapy. The methods disclosed herein address the risks presented by pathogenic diseases in the aforementioned subjects.

The pathogenic disease or disorder may be the result of reinfection or reactivation. The majority of the world's population becomes infected with a pathogen during childhood. After clearance of acute infection, latency is established in the host and may persist for life. Reactivation from latency is associated with various pathologies. For example, cytomegalovirus (CMV) can cause severe disease such as hepatitis following reactivation in immunocompromised hosts. As another example, immunosuppression is known in the art to increase the risk of reactivation of prior infection with Mycobacterium tuberculosis leading to tuberculosis disease. Other pathogenic infections that may lead to a disease or disorder as a result of reinfection or reactivation are known to those of skill in the art, as well as appropriate treatment options, including preventative measures such as vaccinations. For subjects with planned immunosuppression, including the use of corticosteroids, the U.S. Centers for Disease Control and Prevention (CDC) recommends vaccination for Herpes Zoster, S. pneumoniae and Influenza. See, Centers for Disease Control (CDC); Epidemiology and Prevention of Vaccine-Preventable Diseases; The Pink Book.

It has been observed that a subject that received an rAAV as described in the Examples section, developed Herpes Zoster. Without being bound to theory, Herpes Zoster may have developed in the subject that received the rAAV due to the subject having received an immunosuppressant regimen. Herpes Zoster is an infection that results when varicella zoster virus (VZV) reactivates from a latent state. Primary infection of VZV in subjects results in chicken pox, for which even after recovery, VZV remains in the body in a latent state. Subjects with certain conditions such as, without limitation, an immunocompromised state due to a disease or disorder (e.g., cancer, human immunodeficiency virus infection), an immunocompromised state due to bone marrow or solid organ transplant, or taking immunosuppressive medications including corticosteroids, have an increased risk of Herpes Zoster.

As such, methods for reducing the risk of occurrence and/or the severity of transaminitis described herein may result in the development of one or more pathogenic diseases (e.g., diseases associated with a pathogenic infection). In order to reduce the risk of the immunosuppressant regimen being discontinued due to pathogenic infection in the subject, in certain embodiments, a prophylactic vaccine is administered to the subject.

Accordingly, also provided herein are methods for reducing the risk of occurrence and/or the severity of a pathogenic disease in a subject receiving a gene therapy and an immunosuppressant regimen, the method comprising: administering a prophylactic vaccine to a subject that will receive the gene therapy and the immunosuppressant regimen. Such methods are particularly advantageous in that they reduce the risk of the immunosuppressant regimen being discontinued due to pathogenic infection in the subject, and, in turn, reduce the risk that the gene therapy will be ineffective.

It will readily be appreciated by those of skill in the art that a prophylactic vaccine described herein can be one of various types of vaccines known to those of skill in the art. Examples of vaccines known in the art include, without limitation, a live or live-attenuated vaccine, an inactivated vaccine, a subunit vaccine, a toxoid vaccine, and a nucleic acid vaccine. In certain embodiments, the vaccine is a subunit vaccine selected from the group consisting of a polysaccharide vaccine, a conjugate vaccine, a toxoid vaccine, and a recombinant protein vaccine. In certain embodiments, the vaccine is a nucleic acid vaccine selected from the group consisting of a DNA-based vaccine, an RNA-based vaccine, and a recombinant vector vaccine.

Subjects having received an immunosuppressant regimen are said to have altered immunocompetence. Subjects with altered immunocompetence also include those that suffer from primary and/or secondary immunodeficiency that result in a combination of deficits in both humoral and cellular immunity. Subjects with altered immunocompetence have a higher incidence and/or severity of vaccine-preventable diseases. In these subjects, in certain embodiments, administration of live or live-attenuated vaccines must be deferred until a time at which the immune function of the subject has improved, because subjects with altered immunocompetence that receive live vaccines are at an increased risk for adverse reactions caused by the uninhibited growth of the live virus or bacteria. Accordingly, because gene therapy recipients are administrated an immunosuppressant regimen, gene therapy recipients may not be candidates for receiving live or live-attenuated vaccines.

### Varicella Zoster Virus Vaccine Methods

Applicants have determined that there is a risk of reactivation of varicella zoster virus (and subsequent development of herpes zoster) in a subject that is receiving a gene therapy and an accompanying immunosuppressant regimen. The development of herpes zoster in such a subject is highly undesirable because it would necessitate discontinuation of the immunosuppressant regimen, which, in turn, may lead to a failure of the gene therapy. The methods disclosed herein address the risks presented by reactivation of varicella zoster virus in the aforementioned subjects.

Specifically, in one aspect, the instant disclosure provided a method of reducing the risk of occurrence and/or the severity of herpes zoster in a subject receiving a gene therapy and an immunosuppressant regimen. The method generally comprises administering a herpes zoster vaccine to a subject that will receive the gene therapy and immunosuppressant regimen, wherein an initial dose of the herpes zoster vaccine is administered to the subject prior to administration of the immunosuppressant regimen.

Any type of vaccine that is effective at protecting a subject against herpes zoster can be used in the methods disclosed herein. In certain embodiments, the vaccine comprises a polysaccharide, a polypeptide, or a nucleic acid. In certain embodiments, the vaccine is a subunit vaccine. Suitable subunit vaccines include, without limitation, vaccines comprising a varicella zoster virus glycoprotein E antigen. In certain embodiments, the vaccine comprises a recombinant varicella zoster virus glycoprotein E antigen, e.g., a vaccine comprising recombinant varicella zoster virus a glycoprotein E antigen, monophosphoryl lipid A, and the saponin, QS-21. Examples of such recombinant vaccines include, without limitation, the recombinant zoster vaccine sold by GlaxoSmithKline under the name Shingrix.

In general, the zoster vaccine is administered prior to commencement of the immunosuppressant regimen, and/or administration of the gene therapy. The initial dose of the vaccine can be administered at time prior to commencement of the immunosuppressant regimen, once the subject has been identified as a recipient for the gene therapy. In certain embodiments, the initial dose of the vaccine is administered to the subject at least about 1, 2, 3, 4, 5, 6, 7, 8, 9, or 10 weeks prior to commencement of the immunosuppressant regimen.

In general, at least one subsequent dose of the vaccine is administered to the subject after administration of the initial dose. The at least one subsequent dose can be administered at any time after administration of the initial dose. In certain embodiments, the at least one subsequent dose of the vaccine is administered to the subject at least about 1, 2, 3, 4, 5, 6, 7, 8, 9, or 10 weeks prior to commencement of the immunosuppressant regimen.

S. pneumoniae Vaccine Methods

Applicants have determined that there is a risk of reactivation of S. pneumoniae infection in a subject that is receiving a gene therapy and an accompanying immunosuppressant regimen. S. pneumoniae infection can result in, e.g., pneumonia, meningitis, and/or sepsis, in such a subject, and is highly undesirable because it would necessitate discontinuation of the immunosuppressant regimen, which, in turn, may lead to a failure of the gene therapy. The methods disclosed herein address the risks presented by reactivation of S. pneumoniae infection in the aforementioned subjects.

Specifically, in one aspect, the instant disclosure provided a method of reducing the risk of occurrence and/or the severity of an S. pneumoniae related disease or disorder in a subject receiving a gene therapy and an immunosuppressant regimen. The method generally comprises administering a S. pneumoniae vaccine to a subject that will receive the gene therapy and immunosuppressant regimen, wherein an initial dose of the herpes zoster vaccine is administered to the subject prior to administration of the immunosuppressant regimen.

Any type of vaccine that is effective at protecting a subject against S. pneumoniae can be used in the methods disclosed herein. In certain embodiments, the vaccine comprises a polysaccharide, a polypeptide, or a nucleic acid. For example, in certain embodiments, the vaccine is selected from the group consisting of: a live-attenuated vaccine; an inactivated vaccine; a subunit vaccine; a toxoid vaccine; and a nucleic acid vaccine.

In certain embodiments, the vaccine is a subunit vaccine. Suitable subunit vaccines include, without limitation, a polysaccharide vaccine, a conjugate vaccine, a toxoid vaccine, and a recombinant protein vaccine.

In certain embodiments, the vaccine is a conjugate vaccine. Suitable conjugate vaccines include, without limitation, vaccines comprising purified capsular polysaccharides of one or more of serotypes 1, 3, 4, 5, 6A, 6B, 7F, 9V, 14, 18C, 19A, 19F, and 23F of S. pneumoniae conjugated to carrier, e.g., a non-toxic mutant of diphtheria toxin (e.g., CRM197). In certain embodiments, the conjugate vaccine comprises purified capsular polysaccharides of serotypes 1, 3, 4, 5, 6A, 6B, 7F, 9V, 14, 18C, 19A, 19F, and 23F of S. pneumoniae conjugated to CRM197. Examples of such vaccines include PCV13, the pneumococcal conjugate vaccine sold by Merck under the name PREVNAR13®.

In certain embodiments, the vaccine is a polysaccharide vaccine. Suitable polysaccharide vaccines include, without limitation, vaccines comprising purified capsular polysaccharides of one or more of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae. In certain embodiments, the polysaccharide vaccine comprises purified capsular polysaccharides of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae. Examples of such vaccines include PPSV23, the pneumococcal polysaccharide vaccine sold by Pfizer under the name PNEUMOVAX®23.

In general, the S. pneumoniae vaccine is administered prior to commencement of the immunosuppressant regimen, and/or administration of the gene therapy. The initial dose of the vaccine can be administered at any time prior to commencement of the immunosuppressant regimen, once the subject has been identified as a recipient for the gene therapy. In certain embodiments, the initial dose of the vaccine is administered to the subject at least about 1, 2, 3, 4, 5, 6, 7. 8, 9, or 10 weeks prior to commencement of the immunosuppressant regimen.

In general, at least one subsequent dose of the vaccine is administered to the subject after administration of the initial dose. The at least one subsequent dose can be administered at any time after administration of the initial dose. In certain embodiments, the at least one subsequent dose of the vaccine is administered to the subject at least about 1, 2, 3, 4, 5, 6, 7, 8, 9, or 10 weeks prior to commencement of the immunosuppressant regimen.

The initial and subsequent doses of the vaccine can comprise the same vaccine or a different vaccine. In certain embodiments, the initial dose of the vaccine comprises a conjugate vaccine and the at least one subsequent dose of the vaccine comprises a polysaccharide vaccine. In certain embodiments, the initial dose of the vaccine comprises PCV13 and the at least one subsequent dose of the vaccine comprises PPSV23.

### Influenza Vaccine Methods

Applicants have determined that there is a risk of influenza infection in a subject that is receiving a gene therapy and an accompanying immunosuppressant regimen. Influenza infection in such a subject is highly undesirable because it would necessitate discontinuation of the immunosuppressant regimen, which, in turn, may lead to a failure of the gene therapy. The methods disclosed herein address the risks presented by reactivation of Influenza infection in the aforementioned subjects.

Specifically, in one aspect, the instant disclosure provided a method of reducing the risk of occurrence and/or the severity of influenza in a subject receiving a gene therapy and an immunosuppressant regimen. The method generally comprises administering an influenza vaccine to a subject that will receive or has received the gene therapy and immunosuppressant regimen.

Any type of vaccine that is effective at protecting a subject against influenza can be used in the methods disclosed herein. In certain embodiments, the vaccine comprises a polysaccharide, a polypeptide, or a nucleic acid. For example, in certain embodiments, the vaccine is selected from the group consisting of: a live-attenuated vaccine; an inactivated vaccine; a subunit vaccine; a toxoid vaccine; and a nucleic acid vaccine. In certain embodiments, the vaccine is a subunit vaccine. Suitable subunit vaccines include, without limitation, a polysaccharide vaccine, a conjugate vaccine, a toxoid vaccine, and a recombinant protein vaccine.

In certain embodiments, the influenza vaccine is a seasonal influenza vaccine. As known in the art, seasonal influenza vaccines provide narrow protection against select strains of the influenza virus. Seasonal influenza vaccines are needed because strains of influenza change annually, and the efficacy of the influenza vaccine is narrow and short-lived. Viruses that cause influenza include the influenza A virus and the influenza B virus which express a variety of hemagglutinin (H) and neuraminidase (N) antigens. As such, seasonal influenza vaccines are updated annually to better match influenza viruses and their H and N antigens expected to be circulating in a given geographic location. Influenza A viruses are divided into subtypes based on their H and N antigens. Current subtypes of influenza A virus that routinely circulate include subtypes A(H1N1) and A(H3N2). Influenza B viruses are divided into lineages and include B/Yamagata and B/Victoria. Current seasonal influenza vaccines are trivalent or quadrivalent and provide protection against A(H1N1), A(H3N2) and one or two influenza B viruses.

In certain embodiments, the influenza vaccine is a universal influenza vaccine. Such universal influenza vaccines rely on raising immune responses against viral regions that undergo less mutation. For example, a universal influenza vaccine may provide protection via the use of recombinant stalk-specific hemagglutinin, and/or recombinant chimeric hemagglutinin. Other universal influenza vaccines may provide protection via the use of non-hemagglutinin strategies, e.g., via the use of the M2 structural protein.

In certain embodiments, the influenza vaccine is administered prior to commencement of the immunosuppressant regimen, and/or administration of the gene therapy. The vaccine can be administered at time prior to commencement of the immunosuppressant regimen, once the subject has been identified as a recipient for the gene therapy. In certain embodiments, vaccine is administered to the subject at least about 1, 2, 3, 4, 5, 6, 7, 8, 9, or 10 weeks prior to commencement of the immunosuppressant regimen.

In certain embodiments, the influenza vaccine is administered after commencement of the immunosuppressant regimen, and/or administration of the gene therapy. The vaccine can be administered at any time after commencement of the immunosuppressant regimen. In certain embodiments, vaccine is administered to the subject at least about 1, 2, 3, 4, 5, 6, 7, 8, 9, or 10 weeks after commencement of the immunosuppressant regimen.

### III. Gene Therapies

The methods provided herein are generally applicable to any gene therapy method (e.g., liver-directed gene therapy method). For example, in certain embodiments, the gene therapy (e.g., liver-directed gene therapy) is mediated by a recombinant viral vector, e.g., a retroviral vector, a lentiviral vector, an adenoviral vector, and an adeno-associated virus vector.

In general, a recombinant viral vector comprises a transgene. Such transgene can encode, without limitation, a polypeptide or a non-coding RNA (e.g., miRNA, shRNA, siRNA, antisense RNA, gRNA, antagomir, miRNA sponge, RNA aptazyme, RNA aptamer, lncRNA, or ribozyme).

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as α-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2, and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrophin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (I2S), and arylsulfatase A; and mitochondrial proteins, such as frataxin. In certain embodiments, the transgene encodes a polypeptide selected from the group consisting of phenylalanine hydroxylase (PAH), glucost-6-phosphatase (G6Pase), iduronate-2-sulfatase (I2S), arylsulfatase A (ARSA), and frataxin (FXN).

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN6, CLN8, LYST, MYOV, RAB27A, melanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc finger nucleases (ZFN) (see, e.g., Porteus and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effector nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C (Cas8c), type I-D (Cas10d), type I-E (Cse1, Cse2), type I-F (Csy1, Csy2, Csy3), type I-U (GSU0054), type III (Cas10), type III-A (Csm2), type III-B (Cmr5), type III-C (Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, a transcriptional regulatory element (TRE) is operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441), CMV early enhancer/chicken 3-actin (CBA) promoter/rabbit β-globin intron (CAG) (Miyazaki et al. (1989) Gene 79(2): 269-277), CB^{SB} (Jacobson et al. (2006) Molecular Therapy 13(6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32(3): 409-417), mitochondrial heavy-strand promoter (Lodeiro et al. (2012) PNAS 109(17): 6513-6518), ubiquitin promoter (Wulff et al. (1990) FEBS Letters 261:101-105). In certain embodiments, the TRE comprises a cytomegalovirus (CMV) promoter/enhancer, an SV40 promoter, a chicken beta actin (CBA) promoter, an smCBA promoter, a human elongation factor 1 alpha (EF1α) promoter, a minute virus of mouse (MVM) intron which comprises transcription factor binding sites, a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element, a human calmodulin 1 (CALM1) promoter, a human ApoE/C-I hepatic control region (HCR1), a human al-antitrypsin (hAAT) promoter, an extended HCR1, a HS-CRM8 element of an hAAT promoter, a human transthyretin (TTR) promoter, and/or a human Methyl-CpG Binding Protein 2 (MeCP2) promoter. Any of the TREs described herein can be combined in any order to drive efficient transcription. For example, a TRE comprising a CMV enhancer, a CBA promoter, and the splice acceptor from exon 3 of the rabbit beta-globin gene, collectively called a CAG promoter may be used. For example, a TRE comprising a hybrid of CMV enhancer and CBA promoter followed by a splice donor and splice acceptor, collectively called a CASI promoter may be used. For example, a TRE comprising a HCR1 and hAAT promoter may be used.

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the TRE is liver-specific (e.g., hepatocyte-specific). Exemplary liver-specific TREs may comprise one or more elements selected from the group consisting of human albumin promoter, human transthyretin (TTR) promoter, human APOE/C-I hepatic control region (HCR) 1, human APOH promoter, and human SERPINA1 (hAAT) promoter or a hepatic specific regulatory module thereof. In certain embodiments, the liver-specific TRE comprises the TBG SERPINA7 promoter as described in Yan et al. (Gene (2016) 506, 289-294). In certain embodiments, the liver-specific TRE comprises the TBG SERPINA7 promoter as described in Hayashi et al. (Molecular Endocrinology (1993) 7(8), 1049-1060). In certain embodiments, the liver-specific TRE comprises the hAAT SERPINA1 promoter as described in Hafenrichter et al. (Blood (1994) 84(10), 3394-3404). In certain embodiments, the liver-specific TRE comprises the TTR promoter as described in Costa et al. (Molecular and Cellular Biology (1988) 8(1), 81-90). In certain embodiments, the liver-specific TRE comprises the ApoA2 promoter as described in Kan et al. (Nucleic Acids Research (1999) 27(4), 1104-1117). In certain embodiments, the liver-specific TRE comprises the albumin promoter as described in Tang et al. (Biomedical Reports (2017) 6, 627-632). In certain embodiments, the liver-specific TRE comprises the modified fibrinogen promoter as described in Kyostio-Moore et al. (Molecular Therapy (2016) 3, 16006). In certain embodiments, the liver-specific TRE comprises the minimum human APOE/C-I hepatic control region (HCR) 1 promoter as described in Dang et al. (J. Biol. Chem. (1995) 270(38), 22557-85). In certain embodiments, the liver-specific TRE comprises the human APOE/C-I hepatic control region (HCR) 2 promoter as described in Allan et al. (J. Biol. Chem. (1995) 270(44), 26278-81). More liver-specific promoter elements are disclosed in WO 2009/130208 and Kramer et al. (Molecular Therapy (2003) 7, 375-385), which are incorporated by reference herein in their entirety.

In certain embodiments, the recombinant viral vector comprises two or more TREs, optionally comprising at least one of the TREs disclosed above. A skilled person in the art would appreciate that any of these TREs can be combined in any order, and combinations of a constitutive TRE and a tissue-specific TRE can drive efficient and tissue-specific transcription. For example, in certain embodiments, the recombinant viral vector comprises a human HCR1 and a human EF-1α promoter, optionally wherein the human HCR1 is 5′ to the human EF-1α promoter. Similarly, combinations of two or more tissue-specific TREs can drive efficient and tissue-specific transcription. For example, in certain embodiments, the rAAV genome comprises a human HCR1 and an hAAT promoter, optionally wherein the human HCR1 is 5′ to the hAAT promoter. In certain embodiments, the rAAV genome comprises a human HCR1 and an hAAT promoter, optionally wherein the human HCR1 is 5′ to the hAAT promoter. In certain embodiments, the recombinant viral vector comprises a hepatic specific regulatory module of hAAT promoter and a human TTR promoter, optionally wherein the hepatic specific regulatory module is 5′ to the human TTR promoter. In certain embodiments, the rAAV genome comprises a hepatic specific regulatory module of hAAT promoter and a human TTR promoter, optionally wherein the hepatic specific regulatory module is 5′ to the human TTR promoter.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the recombinant viral vector further comprises an intron element. In certain embodiments, the intron element is 5′ to the at least a portion of the transgene. Such intron elements can increase transgene expression, for example, by reducing transcriptional silencing and enhancing mRNA export from the nucleus to the cytoplasm. In certain embodiments, the recombinant viral vector comprises from 5′ to 3′: a TRE, an intron element, and the at least a portion of the transgene.

The intron element can comprise at least a portion of any intron sequence known in the art. In certain embodiments, the intron element is an exogenous intron element (e.g., comprising at least an intron sequence from a different species or a different gene from the same species, and/or a synthetic intron sequence). In certain embodiments, the intron element is an exogenous intron element comprising at least a portion of an intron sequence from a different species. In certain embodiments, the intron element is an exogenous intron element comprising at least a portion of an intron sequence from a different gene from the same species. In certain embodiments, the intron element is an exogenous intron element comprising a synthetic intron sequence. In certain embodiments, the intron element is an exogenous intron element comprising a combination of at least an intron sequence from a different species or a different gene from the same species, and/or a synthetic intron sequence.

A skilled worker will appreciate that intron elements can be designed to mediate RNA splicing by introducing any consensus splicing motifs known in the art (e.g., in Sibley et al., (2016) Nature Reviews Genetics, 17, 407-21, which is incorporated by reference herein in its entirety). Exemplary intron sequences are provided in Lu et al. (2013) Molecular Therapy 21(5): 954-63, and Lu et al. (2017) Hum. Gene Ther. 28(1): 125-34, which are incorporated by reference herein in their entirety. Examples of intron elements include, without limitation, an SV40 intron element and a minute virus of mouse (MVM) intron element. Synthetic intron elements are also known in the art and readily employed by those of skill in the art.

In certain embodiments, the recombinant viral vector comprises a transcription terminator (e.g., a polyadenylation sequence). In certain embodiments, the transcription terminator is 3′ to the at least a portion of the RNA or polypeptide encoded by the transgene. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence. In certain embodiments, the polyadenylation sequence is a bovine growth hormone (BGH) polyadenylation sequence.

In certain embodiments, the recombinant viral vector comprises a transgene operably linked to a TRE. In certain embodiments, the transgene encodes a phenylalanine hydroxylase (PAH). In certain embodiments, the recombinant viral vector comprises a 5′ inverted terminal repeat (5′ ITR) nucleotide sequence 5′ of the TRE and PAH coding sequence, and a 3′ inverted terminal repeat (3′ ITR) nucleotide sequence 3′ of the TRE and PAH coding sequence. The recombinant viral vector can be used to express PAH in any mammalian cells (e.g., human cells).

In certain embodiments, the recombinant viral vector comprises a transgene operably linked to a TRE. In certain embodiments, the recombinant viral vector comprises a transgene operably linked to a TRE. In certain embodiments, the TRE comprises a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24). In certain embodiments, the TRE comprises an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25). In certain embodiments, the TRE comprises an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26). In certain embodiments, the TRE comprises a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 27. In certain embodiments, the recombinant viral vector comprises a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28). In certain embodiments, the recombinant viral vector comprises an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29).

In certain embodiments, the recombinant viral vector comprises a transgene operably linked to a TRE. In certain embodiments, the TRE comprises a composite globin/AIAT intron (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 18). In certain embodiments, the TRE comprises an ApoE-HCR element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 19). In certain embodiments, the TRE comprises an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 20). In certain embodiments, the TRE comprises a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 21. In certain embodiments, the transgene comprises a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the polynucleotide sequence set forth in SEQ ID NO: 22. In certain embodiments, the recombinant viral vector comprises a bovine growth hormone polyadenylation sequence. In certain embodiments, the recombinant viral vector comprises a polynucleotide sequence having at least 80% (e.g., at least 80%, 81%, 82%, 83%, 84%, 85%, 86%, 87%, 88%, 89%, 90%, 91%, 92%, 93%, 94%, 95%, 96%, 97%, 98%, or 99%) sequence identity with the polynucleotide sequence set forth in SEQ ID NO: 23. A recombinant viral vector comprising an ApoE-HCR element, an hAAT promoter, a composite globin/AIAT intron, and a bovine growth hormone polyadenylation sequence is described in U.S. Patent Publication No. US20190376081A1, the disclosure of which is incorporated by reference herein in its entirety.

In certain embodiments, the gene therapy is mediated by a recombinant adeno-associated virus (AAV) vector. For convenience, the present disclosure is further exemplified and described herein by reference to AAV. It would be appreciated by those of skill in the art that the present disclosure is not limited to AAV, and may equally be applied to other gene delivery methods (e.g., gene delivery methods that may trigger an immune response).

In certain embodiments, an AAV vector comprises an AAV capsid comprising an AAV capsid protein, and a recombinant AAV genome. Various AAV capsid proteins are known to those of skill in the art, including, without limitation, a capsid protein from an AAV1, AAV2, AAV3, AAV4, AAV5, AAV6, AAV7, AAV8, AAV9, AAV10, AAV11, or AAV12 serotype. AAV10, AAV11, and AAV12 are described in, e.g., U.S. Patent Publication No. 2003/0138772, the disclosure of which is incorporated by reference herein in its entirety. Other examples of AAV capsid proteins include, without limitation, a capsid protein from AAV-DJ (see, e.g., U.S. Pat. No. 7,588,772, the disclosure of which is incorporated by reference herein in its entirety); AAV-LK03 (see, e.g., U.S. Pat. No. 9,169,299, the disclosure of which is incorporated by reference herein in its entirety); NP59 (see, e.g., U.S. Pat. No. 10,179,176, the disclosure of which is incorporated by reference herein in its entirety); VOY101, VOY201, VOY701, VOY801, VOY1101, AAVPHP.N, AAVPHP.A, AAVPHP.B, PHP.B2, PHP.B3, G2A3, G2B4, G2B5, PHP.S, (see, e.g., PCT Patent Publication No. WO2020/077165, the disclosure of which is incorporated by reference herein in its entirety).

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

In certain embodiments, the gene therapy is mediated by a non-viral gene delivery system. Non-viral gene delivery systems are known in the art and include, without limitation,

In certain embodiments, the gene therapy is a liver-directed gene therapy.

In certain embodiments, the gene therapy is for treating phenylketonuria (PKU). In certain embodiments, the gene therapy comprises an AAV vector comprising an AAV capsid comprising an AAV capsid protein, and a recombinant AAV genome.

In certain embodiments, the AAV capsid protein comprises the amino acid sequence of amino acids 203-736 of SEQ ID NO: 16, and the recombinant AAV genome comprises from 5′ to 3′: a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); and an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29). In certain embodiments, the AAV capsid protein comprises the amino acid sequence of amino acids 138-736 of SEQ ID NO: 16, and the recombinant AAV genome comprises from 5′ to 3′: a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); and an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29). In certain embodiments, the AAV capsid protein comprises the amino acid sequence of amino acids 1-736 of SEQ ID NO: 16, and the recombinant AAV genome comprises from 5′ to 3′: a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); and an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29).

In certain embodiments, the AAV capsid protein comprises the amino acid sequence of amino acids 203-736 of SEQ ID NO: 16, and the recombinant AAV genome comprises from 5′ to 3′: a 5′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 30); a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29); and a 3′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 31). In certain embodiments, the AAV capsid protein comprises the amino acid sequence of amino acids 138-736 of SEQ ID NO: 16, and the recombinant AAV genome comprises from 5′ to 3′: a 5′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 30); a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29); and a 3′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 31). In certain embodiments, the AAV capsid protein comprises the amino acid sequence of amino acids 1-736 of SEQ ID NO: 16, and the recombinant AAV genome comprises from 5′ to 3′: a 5′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 30); a human HCR1 element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 24); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 25); an SV40 intron element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 26); a silently altered human PAH coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 28); an SV40 polyadenylation sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 29); and a 3′ ITR sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 31).

In certain embodiments, the AAV capsid protein comprises the amino acid sequence of amino acids 203-736 of SEQ ID NO: 16, and the recombinant AAV genome comprises SEQ ID NO: 32, 33, or 34. In certain embodiments, the AAV capsid protein comprises the amino acid sequence of amino acids 138-736 of SEQ ID NO: 16, and the recombinant AAV genome comprises SEQ ID NO: 32, 33, or 34. In certain embodiments, the AAV capsid protein comprises the amino acid sequence of amino acids 1-736 of SEQ ID NO: 16, and the recombinant AAV genome comprises SEQ ID NO: 32, 33, or 34.

In certain embodiments, the AAV capsid protein is an AAV5 capsid protein. In certain embodiments, the recombinant AAV genome comprises from 5′ to 3′: an ApoE-HCR element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 19); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 20); a composite globin/AIAT intron (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 18); a codon optimized human phenylalanine hydroxylase coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 22); and a bovine growth hormone polyadenylation sequence. In certain embodiments, the recombinant AAV genome further comprises an AAV2 5′ ITR sequence and an AAV2 3′ ITR sequence. Accordingly, in certain embodiments, the gene therapy for treating PKU comprises an AAV vector comprising an AAV5 capsid protein, and a recombinant AAV genome comprising from 5′ to 3′: an AAV2 5′ ITR sequence; an ApoE-HCR element (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 19); an hAAT promoter (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 20); a composite globin/AIAT intron (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 18); a codon optimized human phenylalanine hydroxylase coding sequence (e.g., a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 22); a bovine growth hormone polyadenylation sequence; and an AAV2 3′ ITR sequence. In certain embodiments, the gene therapy for treating PKU comprises an AAV vector comprising an AAV5 capsid protein, and a recombinant AAV genome comprising a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the polynucleotide sequence set forth in SEQ ID NO: 23. The gene therapy for treating PKU comprising a recombinant AAV genome comprising an ApoE-HCR element, an hAAT promoter, a composite globin/AIAT intron, and a bovine growth hormone polyadenylation sequence is described in U.S. Patent Publication No. US20190376081A1, the disclosure of which is incorporated by reference herein in its entirety.

Also provided is a method of treating a subject having a disease or disorder, the method comprising administering to the subject a liver-directed gene therapy described herein, wherein the subject has received a first immunosuppressant in an initial dosing regimen, and wherein the initial dosing regimen is conducted for at least about 8 weeks. In certain embodiments, the disease or disorder is phenylketonuria (PKU).

### IV. Immunosuppressant Regimens

The methods provided herein are generally applicable to any gene therapy method (e.g., liver-directed gene therapy method) involving an immunosuppressant regimen.

In certain embodiments, liver-directed rAAV delivery to subjects may result in an immune response to the rAAV. In certain embodiments, the immune response to the rAAV is evidenced by an increase in liver function tests. Liver function tests include assaying the levels of certain enzymes and proteins in the blood. Various liver function tests are known in the art including, for example, assaying the level of alanine transaminase (ALT); aspartate transaminase (AST); alkaline phosphatase (ALP); albumin and total protein; bilirubin; gamma-glutamyltransferase (GGT); and L-lactate dehydrogenase (LD), and assaying the time it takes blood to clot (also known in the art as prothrombin time (PT)). In certain embodiments, the immune response to the rAAV is evidenced by an increase in AAV capsid-specific T cells in the peripheral blood. As known in the art, capsid-specific CD8+ T cell responses peak within 4-8 weeks and are coincident with elevations in serum ALT and AST and elimination of AAV-transduced cells. Further, humoral immunity to the AAV capsid has been shown to occur within 2-4 weeks of viral delivery. In certain embodiments, elevated transaminases (e.g., elevated levels of ALT and AST) are observed in subjects, and may be self-limited and unaccompanied by additional signs of liver toxicity. The elevation of transaminases (e.g., ALT and AST) can be controlled by the administration of anti-inflammatory and/or immunosuppressive therapies (e.g., an immunosuppressant regimen). Accordingly, provided herein are methods for reducing the risk of occurrence and/or severity of transaminitis in a subject receiving a gene therapy (e.g., a liver-directed rAAV).

In certain embodiments, the subject is administered a prophylactic immunosuppressant regimen (e.g., a prophylactic prednisolone regimen) before administration of the rAAV of the present disclosure. In certain embodiments, the subject is administered a prophylactic immunosuppressant regimen one day before administration of the rAAV of the present disclosure. In certain embodiments, the subject is administered a prophylactic immunosuppressant regimen therapy during administration of the rAAV of the present disclosure. In certain embodiments, the subject is administered a prophylactic immunosuppressant regimen after administration of the rAAV of the present disclosure. In certain embodiments, the subject is administered a prophylactic immunosuppressant regimen before, during, and/or after administration of the rAAV of the present disclosure. A prophylactic immunosuppressant regimen described herein may limit the immunologic response in the liver and to maintain PAH expression and prevent loss of vector.

In certain embodiments, the duration and doses of the immunosuppressant regimen are intended to cover the anticipated period of peak immune response (e.g., about 8 weeks following viral delivery), followed by a gradual taper. It will be readily apparent to those of skill in the art (e.g., an attending physician) that administration of the immunosuppressant regimen, including gradual taper thereof, will depend on the general tolerability of the subject to the immunosuppressant regimen.

The route of administration of the immunosuppressant regimen will readily be able to be determined by those of skill in the art. In certain embodiments, the immunosuppressant regimen will be administered orally. In certain embodiments, the immunosuppressant regimen will be administered systemically (e.g., via intravenous or parenteral routes).

Suitable immunosuppressant regimens are known in the art and include use of corticosteroids. Accordingly, in certain embodiments, the subject is administered a corticosteroid (e.g., prednisolone) before, during, and/or after administration of the rAAV of the present disclosure. In certain embodiments, the corticosteroid is glucocorticoid. Examples of corticosteroids include, without limitation, hydrocortisone, cortisone, ethamethasoneb, prednisone, prednisolone, triamcinolone, methylprednisolone, and dexamethasone. In certain embodiments, the subject is administered prednisolone before, during, and/or after administration of the rAAV of the present disclosure. In certain embodiments, the subject is administered dexamethasone before, during, and/or after administration of the rAAV of the present disclosure.

In certain embodiments, the immunosuppressant regimen (e.g., prednisolone regimen) is administered to the subject before administration of the rAAV of the present disclosure. In certain embodiments, the immunosuppressant regimen (e.g., prednisolone regimen) is administered to the subject at least one day, at least two days, at least three days, at least four days, at least five days, at least six days, at least seven days, at least eight days, at least nine days, or at least ten days, before administration of the rAAV of the present disclosure. In certain embodiments, the immunosuppressant regimen (e.g., prednisolone regimen) is administered to the subject one day before administration of the rAAV of the present disclosure.

In certain embodiments, the immunosuppressant regimen (e.g., prednisolone regimen) is administered to the subject for about 20 or about 21 weeks. In certain embodiments, the immunosuppressant regimen (e.g., prednisolone regimen) is administered to the subject for at least about one week, at least about two weeks, at least about three weeks, at least about four weeks, at least about five weeks, at least about six weeks, at least about seven weeks, at least about eight weeks, at least about nine weeks, at least about ten weeks, at least about eleven weeks, at least about twelve weeks, at least about thirteen weeks, at least about fourteen weeks, at least about fifteen weeks, at least about sixteen weeks, at least about seventeen weeks, at least about eighteen weeks, at least about nineteen weeks, at least about twenty weeks, at least about twenty one weeks, or at least about twenty two weeks. It will be appreciated by those of skill in the art that the duration of the immunosuppressant regimen described herein may depend on the tolerability of the subject to the therapy, and or depend on clinical developments during the course of the therapy. For example, in certain embodiments, if at any time during the immunosuppressant regimen, the subject exhibits an abnormal result of a liver function test (e.g., exhibits an abnormal serum level of one or more liver enzymes), the immunosuppressant regimen may be restarted and/or modified to include additional doses for additional durations. In such embodiments the duration of the immunosuppressant regimen will be prolonged, e.g., beyond the about 20 week duration.

In certain embodiments, the immunosuppressant regimen (e.g., prednisolone or dexamethasone regimen) is administered at doses described in prednisolone equivalents. As used herein, the term “prednisolone equivalent” refers to a dose of a corticosteroid that results in substantially the same effect as the effect of a certain dose of prednisolone (e.g., immunosuppressive effect of a certain dose of prednisolone) when administered to a subject. For example, a 5 mg dose of prednisolone is known in the art to elicit substantially the same effect as 0.75 mg of dexamethasone when administered to a subject. As such, a 5 mg dose in prednisolone equivalents is readily understood by those of skill in the art to encompass a 5 mg dose of prednisolone, or a 0.75 mg dose of dexamethasone. Corticosteroid equivalency conversion tables are available to and accessible by those of skill in the art and can be readily accessed from, for example, a clinical decision support resource such as UpToDate. In certain embodiments, the duration and doses of the immunosuppressant regimen are calculated according to the corticosteroid equivalency conversion values as shown in Table 1.

TABLE 1
Corticosteroid Equivalent Doses

| Corticosteroid | Equivalent dose (mg) |
|---|---|
| Hydrocortisone (cortisol) | 20 |
| Cortisone acetate | 25 |
| Prednisone | 5 |
| Prednisolone | 5 |
| Methylprednisolone | 4 |
| Triamcinolone | 4 |
| Dexamethasone | 0.75 |
| Betamethasone | 0.6 |

In certain embodiments, the immunosuppressant regimen (e.g., prednisolone or dexamethasone regimen) is administered at a certain dose per day. For example, the therapy can comprise a dose in mg per day. In certain embodiments, the immunosuppressant regimen (e.g., prednisolone or dexamethasone regimen) is administered according to a weight-based dose, e.g., at a certain dose according to the weight of a subject, per day. For example, the therapy can comprise a dose in mg per kg of a subject per day.

In certain embodiments, the immunosuppressant regimen (e.g., prednisolone regimen) is administered at about 1 mg/day to about 100 mg/day. In certain embodiments, the immunosuppressant regimen (e.g., prednisolone regimen) is administered at about 1 mg/day to about 100 mg/day in prednisolone equivalents. In certain embodiments, the immunosuppressant regimen (e.g., prednisolone regimen) is administered at about 1 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, or about 100 mg/day. In certain embodiments, the immunosuppressant regimen (e.g., prednisolone regimen) is administered at about 1 mg/day, about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, or about 100 mg/day in prednisolone equivalents.

In certain embodiments, specific doses of the immunosuppressant regimen are administered according to a sequential dosing regimen. In certain embodiments, the immunosuppressant regimen is administered according to a sequential dosing regimen. In certain embodiments, the immunosuppressant regimen is administered according to the following sequential dosing regimen: about 60 mg/day in prednisolone equivalents for about two weeks; about 40 mg/day in prednisolone equivalents for about six weeks; about 30 mg/day in prednisolone equivalents for about three weeks; about 20 mg/day in prednisolone equivalents for about three weeks; about 10 mg/day in prednisolone equivalents for about five weeks; and about 5 mg/day in prednisolone equivalents for about one week. In certain embodiments, the final step of about 5 mg/day in prednisolone equivalents for about one week may be continued for a total of about two weeks. In certain embodiments, specific doses of the immunosuppressant regimen are administered according to a sequential dosing regimen. In certain embodiments, the prednisolone is administered according to a sequential dosing regimen. In certain embodiments, the prednisolone is administered according to the following sequential dosing regimen: about 60 mg/day for about two weeks; about 40 mg/day for about six weeks; about 30 mg/day for about three weeks; about 20 mg/day for about three weeks; about 10 mg/day for about five weeks; and about 5 mg/day for about one week. In certain embodiments, the final step of about 5 mg/day for about one week may be continued for a total of about two weeks.

In certain embodiments, the prednisolone is administered according to the following sequential dosing regimen, wherein the sequential dosing regimen employs a higher dose for the initial eight weeks: about 60 mg/day for about eight weeks; about 40 mg/day for about four weeks; about 30 mg/day for about three weeks; about 20 mg/day for about two weeks; about 10 mg/day for about two weeks; and about 5 mg/day for about one week. In certain embodiments, the final step of about 5 mg/day for about one week may be continued for a total of about two weeks. In certain embodiments, following the initial about eight weeks prednisolone treatment, prednisolone taper below about 60 mg/day is started if the ALT and/or AST levels of the subject are within normal range, provided there are no tolerability issues. In certain embodiments, following the initial about eight weeks prednisolone treatment, prednisolone taper below about 60 mg/day is started if the ALT and/or AST levels of the subject are within baseline levels, provided there are no tolerability issues.

In certain embodiments, the immunosuppressant regimen is administered at about 0.08 mg/kg/day in prednisolone equivalents to about 1 mg/kg/day in prednisolone equivalents. In certain embodiments, the immunosuppressant regimen is administered at about 0.05 mg/kg/day, about 0.08 mg/kg/day, about 0.10 mg/kg/day, about 0.12 mg/kg/day, about 0.15 mg/kg/day, about 0.17 mg/kg/day, about 0.20 mg/kg/day, about 0.22 mg/kg/day, about 0.25 mg/kg/day, about 0.28 mg/kg/day, about 0.30 mg/kg/day, about 0.33 mg/kg/day, about 0.35 mg/kg/day, about 0.37 mg/kg/day, about 0.40 mg/kg/day, about 0.42 mg/kg/day, about 0.45 mg/kg/day, about 0.48 mg/kg/day, about 0.50 mg/kg/day, about 0.52 mg/kg/day, about 0.55 mg/kg/day, about 0.58 mg/kg/day, about 0.60 mg/kg/day, about 0.62 mg/kg/day, about 0.65 mg/kg/day, about 0.67 mg/kg/day, about 0.70 mg/kg/day, about 0.72 mg/kg/day, about 0.75 mg/kg/day, about 0.78 mg/kg/day, about 0.80 mg/kg/day, about 0.82 mg/kg/day, about 0.85 mg/kg/day, about 0.88 mg/kg/day, about 0.90 mg/kg/day, about 0.92 mg/kg/day, about 0.95 mg/kg/day, about 0.98 mg/kg/day, about 1 mg/kg/day, about 1.02 mg/kg/day, about 1.05 mg/kg/day, about 1.08 mg/kg/day, about 1.1 mg/kg/day in prednisolone equivalents. In certain embodiments, the immunosuppressant regimen comprises administration of dexamethasone at about 0.0125 mg/kg/day to about 0.15 mg/kg/day. In certain embodiments, the immunosuppressant regimen comprises administration of dexamethasone at about 0.008 mg/kg/day, about 0.009 mg/kg/day, about 0.01 mg/kg/day, about 0.0125 mg/kg/day, about 0.015 mg/kg/day, about 0.02 mg/kg/day, about 0.025 mg/kg/day, about 0.03 mg/kg/day, about 0.035 mg/kg/day, about 0.04 mg/kg/day, about 0.045 mg/kg/day, about 0.05 mg/kg/day, about 0.055 mg/kg/day, about 0.06 mg/kg/day, about 0.065 mg/kg/day, about 0.07 mg/kg/day, about 0.075 mg/kg/day, about 0.08 mg/kg/day, about 0.085 mg/kg/day, about 0.09 mg/kg/day, about 0.095 mg/kg/day, about 0.1 mg/kg/day, about 0.105 mg/kg/day, about 0.110 mg/kg/day, about 0.115 mg/kg/day, about 0.120 mg/kg/day, about 0.125 mg/kg/day, about 0.130 mg/kg/day, about 0.135 mg/kg/day, about 0.140 mg/kg/day, about 0.145 mg/kg/day, about 0.150 mg/kg/day, about 0.155 mg/kg/day, about 0.160 mg/kg/day.

In certain embodiments, specific doses of immunosuppressant regimen are administered according to a weight-based sequential dosing regimen. In certain embodiments, the dexamethasone is administered according to a weight-based sequential dosing regimen. In certain embodiments, the dexamethasone is administered according to the following sequential dosing regimen, wherein the sequential dosing regimen employs a higher dose for the initial eight weeks: (1) about 0.15 mg/kg/day for eight weeks; (2) about 0.1 mg/kg/day for four weeks; (3) about 0.075 mg/kg/day for three weeks; (4) about 0.05 mg/kg/day for two weeks; (5) about 0.025 mg/kg/day for two weeks; and (6) about 0.0125 mg/kg/day for one week. In certain embodiments, following the initial eight weeks of dexamethasone treatment (step (1)), dexamethasone taper below 0.15 mg/kg/day is started if the ALT and AST levels of the subject are within normal range, provided there are no tolerability issues. In certain embodiments, the sequential dosing regimen employs a slower taper at step (5) or (6). In certain embodiments, step (5) or (6) is replaced with prednisolone at about 10 mg/day or prednisolone at about 5 mg/day, respectively. Accordingly, in certain embodiments, the immunosuppressant regimen is administered according to the following sequential dosing regimen: (1) about 0.15 mg/kg/day dexamethasone for eight weeks; (2) about 0.1 mg/kg/day dexamethasone for four weeks; (3) about 0.075 mg/kg/day dexamethasone for three weeks; (4) about 0.05 mg/kg/day dexamethasone for two weeks; (5) about 10 mg/day prednisolone for two weeks; and (6) about 5 mg/day prednisolone for one week. In certain embodiments, where a subject whose transaminase values rise during step (1) or step (2) dexamethasone doses, a higher dexamethasone dose is administered to the subject, e.g., 0.3 mg/kg/day dexamethasone.

In certain embodiments, a subject experiences elevated AST and/or ALT levels greater than 1.5 times the upper limit of normal during the prophylactic immunosuppressant regimen described herein. In certain embodiments, a subject experiences elevated AST and/or ALT levels greater than 2 times the upper limit of normal during the prophylactic immunosuppressant regimen described herein. In such embodiments, the immunosuppressant regimen will be re-escalated or re-started at a certain dose.

Accordingly, in certain embodiments, where a subject experiences elevated levels of AST and/or ALT greater than 1.5 times the upper limit of normal (e.g., greater than 2 times ULN) during a prophylactic prednisolone sequential dosing regimen, for example, about 60 mg/day for two weeks; about 40 mg/day for six weeks; about 30 mg/day for three weeks; about 20 mg/day for three weeks; about 10 mg/day for five weeks; and about 5 mg/day for one or two weeks, the prednisolone will be re-escalated or re-started at 60 mg/day, and then tapered again, according to the same sequential dosing regimen, e.g., about 60 mg/day for two weeks; about 40 mg/day for six weeks; about 30 mg/day for three weeks; about 20 mg/day for three weeks; about 10 mg/day for five weeks; and about 5 mg/day for one or two weeks.

Accordingly, in certain embodiments, where a subject experiences elevated levels of AST and/or ALT greater than 1.5 times the upper limit of normal (e.g., greater than 2 times ULN) during a prophylactic prednisolone sequential dosing regimen, for example, where the sequential dosing regimen employs a higher dose for the initial eight weeks: about 60 mg/day for eight weeks; about 40 mg/day for four weeks; about 30 mg/day for three weeks; about 20 mg/day for two weeks; about 10 mg/day for two weeks; and about 5 mg/day for one or two weeks, the prednisolone will be re-escalated or re-started at 60 mg/day, and then tapered again, according to the same prednisolone sequential dosing regimen, e.g., about 60 mg/day for eight weeks; about 40 mg/day for four weeks; about 30 mg/day for three weeks; about 20 mg/day for two weeks; about 10 mg/day for two weeks; and about 5 mg/day for one or two weeks. In certain embodiments, following the initial eight weeks of high dose prednisolone treatment, prednisolone taper below 60 mg/day is started if the ALT and AST levels of the subject are within normal range, provided there are no tolerability issues.

Accordingly, in certain embodiments, where a subject experiences elevated levels of AST and/or ALT greater than 1.5 times the upper limit of normal during a prophylactic dexamethasone sequential dosing regimen, for example, where the sequential dosing regimen employs a higher dose for the initial eight weeks: (1) about 0.15 mg/kg/day for eight weeks; (2) about 0.1 mg/kg/day for four weeks; (3) about 0.075 mg/kg/day for three weeks; (4) about 0.05 mg/kg/day for two weeks; (5) about 0.025 mg/kg/day for two weeks; and (6) about 0.0125 mg/kg/day for one week, the dexamethasone will be re-escalated or re-started at 0.15 mg/kg/day, and then tapered again, according to the same dexamethasone sequential dosing regimen, e.g., (1) about 0.15 mg/kg/day for eight weeks; (2) about 0.1 mg/kg/day for four weeks; (3) about 0.075 mg/kg/day for three weeks; (4) about 0.05 mg/kg/day for two weeks; (5) about 0.025 mg/kg/day for two weeks; and (6) about 0.0125 mg/kg/day for one week. In certain embodiments, following the initial eight weeks of dexamethasone treatment (step (1)), dexamethasone taper below 0.15 mg/kg/day is started if the ALT and AST levels of the subject are within normal range, provided there are no tolerability issues. In certain embodiments, the sequential dosing regimen employs a slower taper at step (5) or (6). In certain embodiments, step (5) or (6) is replaced with prednisolone at about 10 mg/day or prednisolone at about 5 mg/day, respectively.

In certain embodiments, a subject experiences elevated levels of AST and/or ALT greater than 1.5 times the upper limit of normal following the end of a prophylactic immunosuppressant regimen described herein. In certain embodiments, a subject experiences elevated levels of AST and/or ALT greater than 2 times the upper limit of normal following the end of a prophylactic immunosuppressant regimen described herein. In such embodiments, the immunosuppressant regimen will be re-started according to a modified regimen.

Accordingly, in certain embodiments, where a subject experiences elevated levels of AST and/or ALT greater than 1.5 times the upper limit of normal (e.g., greater than 2 times ULN) following the end of a prophylactic prednisolone sequential dosing regimen, for example, about 60 mg/day for two weeks; about 40 mg/day for six weeks; about 30 mg/day for three weeks; about 20 mg/day for three weeks; about 10 mg/day for five weeks; and about 5 mg/day for one or two weeks, the prednisolone will be re-started according to the following dosing regimen: about 60 mg/day for two weeks or until ALT and AST levels have declined to below or about the subject's baseline levels; about 40 mg/day for two weeks; about 30 mg/day for two weeks; about 20 mg/day for two weeks; about 10 mg/day for two weeks; and about 5 mg/day for two weeks. In certain embodiments, the taper below the about 60 mg/day prednisolone for two weeks does not start until the ALT and AST levels have declined to the subject's baseline levels, provided that the subject tolerates the regimen. In certain embodiments, the duration of the about 60 mg/day prednisolone dose does not exceed four weeks.

Accordingly, in certain embodiments, where a subject experiences elevated levels of AST and/or ALT greater than 1.5 times the upper limit of normal (e.g., greater than 2 times ULN) following the end of a prophylactic dexamethasone sequential dosing regimen, for example, about 60 mg/day for two weeks; (1) about 0.15 mg/kg/day for eight weeks; (2) about 0.1 mg/kg/day for four weeks; (3) about 0.075 mg/kg/day for three weeks; (4) about 0.05 mg/kg/day for two weeks; (5) about 0.025 mg/kg/day for two weeks; and (6) about 0.0125 mg/kg/day for one week, the dexamethasone will be re-started according to the following dosing regimen: (1) about 0.15 mg/kg/day for two weeks; (2) about 0.1 mg/kg/day for two weeks; (3) about 0.075 mg/kg/day for two weeks; (4) about 0.05 mg/kg/day for two weeks; (5) about 0.025 mg/kg/day for two weeks; and (6) about 0.0125 mg/kg/day for two weeks.

### Other Concomitant Therapy

In certain embodiments, subjects will continue their usual dietary regimen. In certain embodiments, the baseline diet will be established, and may be defined as +25% of average total protein intake (intact and medical), whether Phe-restricted or unrestricted. In certain embodiments, the baseline diet will be maintained following administration of the rAAV of the present disclosure. In certain embodiments, modification of the diet may be made based on: (1) at 8 weeks, if three Phe values during the first 8 weeks are ≤360 μmol/L; and/or (2) prior to 8 weeks, if three Phe values during the first 8 weeks (measured at least one week apart) are <120 μmol/L.

In certain embodiments, subjects taking medications for the treatment of ADHD, depression, anxiety, or other psychiatric disorders at study entry must be on a stable dose for ≥8 weeks prior to administration of the rAAV of the present disclosure, and may continue with the same dose regimen throughout the study, unless otherwise determined by a physician for medical reasons.

In certain embodiments, use of any medications for PKU, including Kuvan®, LNAA, and Palynziq™, may be prohibited unless the plasma Phe concentration is considered to be unsafe for the subject, and it is determined that such treatment is medically necessary following modification of diet.

### EXAMPLES

The following examples are offered by way of illustration, and not by way of limitation.

### Example 1: Human PAH Transfer Vector

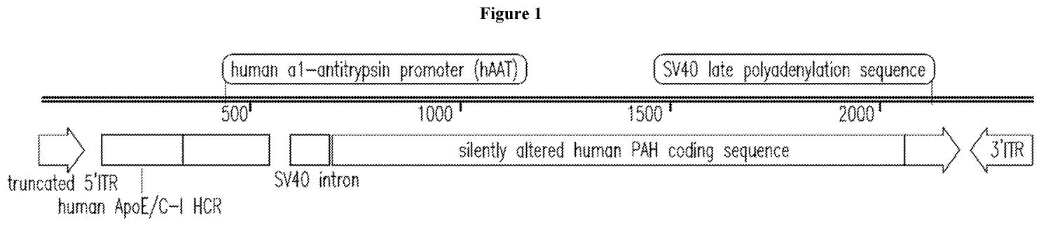

The AAV gene transfer vector pHMI-hPAH-TC-025, as shown in FIG. 1, comprises 5′ to 3′ the following genetic elements: a truncated 5′ ITR element, a human hepatic control region 1 (HCR1), a human al-antitrypsin (hAAT) promoter, an SV4 intron, a silently altered human PAH coding sequence, an SV40 polyadenylation sequence, and a modified 3′ ITR element. The sequences of these elements are set forth in Table 2. The truncated 5′ ITR allows the vector to form a double-stranded AAV genome after transduction into cells. This vector is capable of expressing a human PAH protein in a human hepatocyte.

TABLE 2
Genetic Elements in Human PAH Transfer
Vector pHMI-hPAH-TC-025

| Genetic Element | SEQ ID NO |
|---|---|
| truncated 5′ ITR element | 30 |
| human HCR1 | 24 |
| human α1-antitrypsin (hAAT) promoter | 25 |
| SV40 intron | 26 |
| transcriptional regulatory region comprising the human | 27 |
| HCR1 and hAAT promoter | |
| codon-altered human PAH coding sequence | 28 |
| SV40 polyadenylation sequence | 29 |
| modified 3′ ITR element | 31 |
| transfer genome (from HCR1 to polyadenylation | 32 |
| sequence) | |
| transfer genome (from 5′ ITR to 3′ ITR) | 33 |
| full sequence of transfer vector | 34 |

### Example 2: Efficacy of a PAH Transfer Vector in a Mouse Model of PKU

Pah^{-/-} (PAH^{enu2}) mice were housed in clear polycarbonate cages with contact bedding in an isolator. PicoLab Mouse Diet 5058 was provided to the animals ad libitum. Spring or tap water acidified with 1N HCl to a targeted pH of 2.5-3.0 was provided ad libitum. Vectors packaged in AAVHSC15 capsid were prepared in PBS (with Ca and Mg), supplemented with 35 mM NaCl, 1% sucrose, and 0.05% Pluronic F-68. The formulation was injected intravenously via the tail vein.

Blood samples were collected every week after the administration of the PAH transfer vector (0 week: prior to administration) by facial vein puncture or tail snip. The samples were allowed to clot at room temperature for at least 30 minutes, centrifuged at ambient temperature at minimum 1000×g for 10 minutes and the serum samples were extracted. Serum samples were stored at −70° C. Serum phenylalanine and tyrosine levels were measured by tandem mass spectrometry.

For collection of tissue samples, the animals underwent cardiac perfusion with saline. Liver (caudate lobe), kidney (left), brain, heart, and muscle (quadriceps) tissues were snap frozen in liquid nitrogen and stored at −70° C. The snap frozen tissues were ground into powder in liquid nitrogen in a mortar and pestle and divided into aliquots to test for PAH expression for vector genome biodistribution by qPCR.

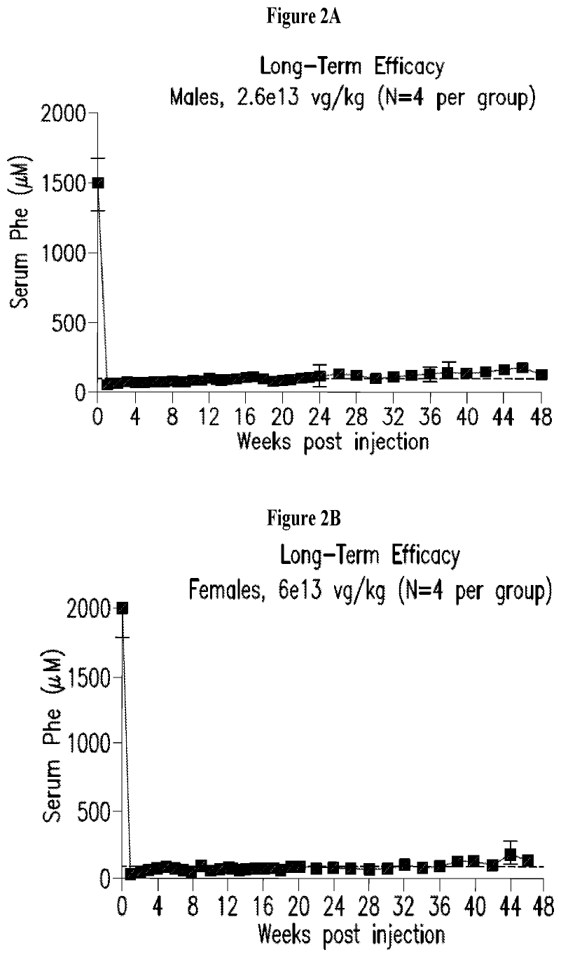

To examine the long-term efficacy of an rAAV comprising pHMI-hPAH-TC-025 packaged in AAVHSC15 capsid, a single dose of 2.6×10¹³ vector genomes per kg of body weight was administered to male Pah^{-/-} (PAHe^{enu2}) mice, and a single dose of 6×10¹³ vector genomes per kg of body weight was administered to female Pah^{-/-} (PAH^{enu2}) mice. As shown in FIGS. 2A and 2B, the administration of the pHMI-hPAH-TC-025-containing rAAV led to significant reduction of Phe levels within one week. This reduction persisted for at least 48 weeks in male mice, and at least 46 weeks in female mice. An increase of PAH mRNA was observed by ddPCR in the liver samples of these mice collected 4 weeks post injection relative to the mice not administered the rAAV vector. An increase of the PAH enzymatic activity was also detected in liver samples by mass spectrometry.

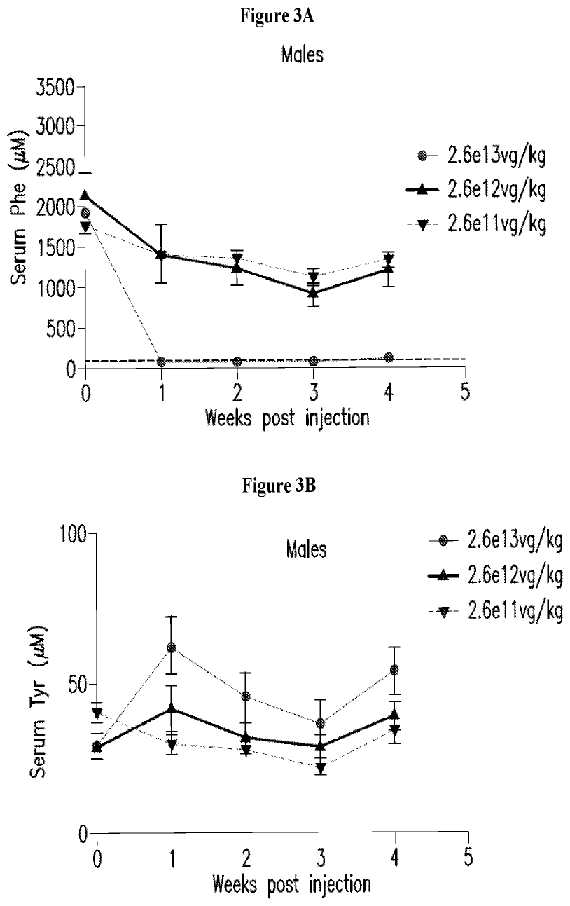

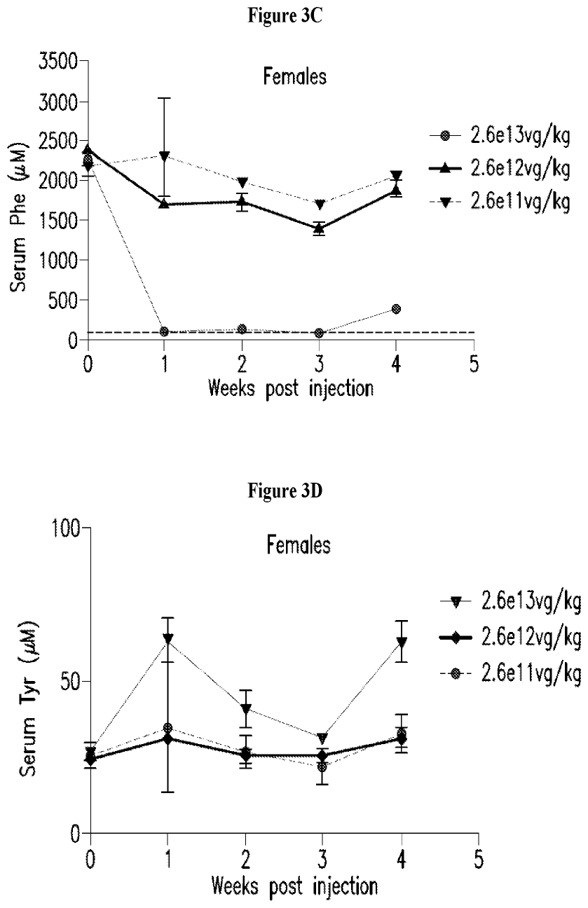

The efficacy of different doses of the pHMI-hPAH-TC-025-containing rAAV described above was further assessed. A single dose of 2.6×10¹¹, 2.6×10¹², or 2.6×10¹³ vector genomes per kg of body weight was administered to male mice and female Pah^{-/-} (PAH^{enu2}) mice, and the serum levels of Phe and Tyr were measured. As shown in FIGS. 3A-3D, the dose of 2.6×10¹³ vector genomes per kg of body weight reduced the Phe levels and increased the Tyr levels more significantly than the two lower doses, and maintained complete reduction of serum Phe levels during the time examined in both male and female subjects.

### Example 3: Clinical Study of a PAH Transfer Vector

This example describes the protocol for a Phase 1/2, randomized, concurrently-controlled, dose escalation study to evaluate the safety and efficacy of an rAAV comprising the pHMI-hPAH-TC-025 vector packaged in AAVHSC15 capsid, in adult phenylketonuria (PKU) subjects with PAH deficiency.

Subjects will undergo screening assessments prior to study entry, with the screening period lasting up to 45 days. Prior to administration of pHMI-hPAH-TC-025 vector packaged in AAVHSC15 capsid, subjects will be admitted to the clinical research unit and prophylactic steroid administration will be initiated. One approximately 120-minute administration of pHMI-hPAH-TC-025 vector packaged in AAVHSC15 capsid will occur in a clinical research unit setting. Subjects may be discharged home after they have been observed for at least 24 hours in the clinical research unit following administration of pHMI-hPAH-TC-025 vector packaged in AAVHSC15 capsid, provided they are clinically stable. Subjects will continue their usual dietary regimen (either Phe-restricted or unrestricted diet) during the screening period and following administration of pHMI-hPAH-TC-025 vector packaged in AAVHSC15 capsid.

Subjects will undergo safety and efficacy observation for 52 weeks in this study following administration of pHMI-hPAH-TC-025 vector packaged in AAVHSC15 capsid. Up to three dose levels of pHMI-hPAH-TC-025 vector packaged in AAVHSC15 capsid will be investigated. All doses are anticipated to provide a clinically relevant decrease in plasma Phe concentration. At a given dose level, 2 subjects will be enrolled and dosed initially. Dosing of the first 2 subjects in each cohort will be staggered, with at least a 21-day interval between dosing of each subject. At least 21-day safety follow-up and Phe concentration data for each subject will be reviewed before the subsequent subject is dosed in that cohort.

Following evaluation of at least 21 days of data from the first 2 subjects in a cohort, a decision can be made to either: (1) escalate to the next dose level, (2) add one additional subject to the same cohort, or (3) expand the cohort at the selected dose to enroll up to 9 additional subjects. 6 subjects will be randomized to receive pHMI-hPAH-TC-025 vector packaged in AAVHSC15 capsid and 3 subjects will be randomized to a concurrent delayed treatment control arm.

Decisions regarding dose escalation and expansion will be based on safety and changes in plasma Phe concentrations relative to the treatment guidelines for PKU. Treatment guidelines describe the range of 120-360 mol/L as PAH deficiency not requiring treatment, and the normal range of blood Phe (e.g., plasma Phe) for an individual without hyperphenylalaninemia (TWA) or PKU as 58±±14 (SD) μmol/L.

### Inclusion and Exclusion Criteria

Suitable subjects for the study will be enrolled according to the inclusion and exclusion criteria set forth in Table 3.

TABLE 3
Inclusion and Exclusion Criteria
Inclusion Criteria:
Subject is able to understand the purpose and risks of the study, is willing to provide
informed consent, and is able to comply with all study procedures and 4-year long-
term follow up.
Adults 18-55 years of age at the time of informed consent.
Diagnosis of classic PKU (due to PAH deficiency).
Two plasma Phe values with a concentration of ≥600 μmol/L drawn at least 48 hours
apart during the screening period and at least one historical value ≥600 μmol/L in
the preceding 12 months.
Subject has the ability and willingness to maintain their baseline diet (±25% of
average total protein intake (intact and medical), whether Phe-restricted or
unrestricted, as established during the 45-day screening period) after administration
of pHMI-hPAH-TC-025 vector packaged in AAVHSC15 capsid, unless otherwise
directed.
If applicable, ability to maintain stable dose of medication for attention-
deficit/hyperactivity disorder (ADHD), depression, anxiety, or other psychiatric
disorder for >8 weeks prior to enrollment and willing to maintain stable dose
throughout study unless a change is medically indicated.
Males and Females of childbearing potential must be willing to use effective
contraception for 12 months following administration of pHMI-hPAH-TC-025
vector packaged in AAVHSC15 capsid which includes barrier contraception (male
or female condom) during the 6 months after administration.
Exclusion Criteria:
Subjects with PKU that is not due to PAH deficiency.
Presence of anti-AAVHSC15 neutralizing antibody (at titer >1:5).
History of or positive test result for human immunodeficiency virus (HIV).
History of or positive test result for hepatitis C virus antibody or hepatitis B virus
(defined as positive for both hepatitis B surface antigen and hepatitis B core
antibody), or current treatment with an antiviral therapy for hepatitis B or C.
History of significant underlying liver disease, liver transplant, genetic liver disease,
cirrhosis, NASH, or other liver condition that would preclude participation in the
study as determined by the investigator.
History of drug abuse or alcoholism that would limit participation in the study, as
determined by the investigator or subjects who exceed moderate drinking levels
defined as: >4 drinks on any single day or >14 drinks per week if male; >3 drinks on
any single day or >7 drinks per week if female.
ALT >1.5× ULN and AST >1.5× ULN.
Alkaline phosphatase >1.5× ULN.
Total bilirubin >1.5× ULN, direct bilirubin ≥1.5× ULN.
Serum creatinine >1.5× ULN.
Hematology values outside of the normal range (hemoglobin <11.0 g/dL for males
or <10.0 g/dL for females; white blood cells (WBC) <3,000 μL; absolute neutrophils
<1500/μL; platelets <100,000 μL).
Hemoglobin A1c >7.9% or fasting glucose >200 mg/dL.
Any clinically significant abnormal laboratory result at screening, in the opinion of
the Investigator.
Contraindication to corticosteroid use or conditions that could worsen in the presence
of corticosteroids, as assessed and determined by the Investigator.
Previously received gene therapy for the treatment of any condition.
Subject is pregnant, breastfeeding, or intends to become pregnant during the study
period.
Scheduled or anticipated major surgery in the 12 weeks following investigational
gene therapy infusion for this study.
Use in past 30 days of levodopa.
Use of any investigational products within 30 days prior to screening.
Current enrollment in any other investigational study.
Presence of an untreated or inadequately treated active infection or an infection
requiring systemic antiviral or antimicrobial therapy at any time during the screening
period.
Use of any medication that is intended to treat PKU, including the use of large neutral
amino acids (LNAAs), within 30 days prior to administration of study drug.
Current body mass index (BMI) ≥35 kg/m² (excludes Obesity Classes II and III).
Weight >100 kg.
Have a clinically significant medical condition that in the investigator's opinion
would pose an unnecessary risk (including history of chronic infection such as HIV
or other chronic diseases), limit the participation of the subject in the study or impact
the ability to interpret study results.
Has a malignancy, or a history of malignancy, with the exception of successfully
treated basal or squamous cell carcinoma of the skin.
Any other condition that would not allow the potential subject to complete follow-up
examinations during the course of the study or, in the opinion of the investigator,
makes the potential subject unsuitable for the study.

### Investigational Product, Dosage, and Mode of Administration

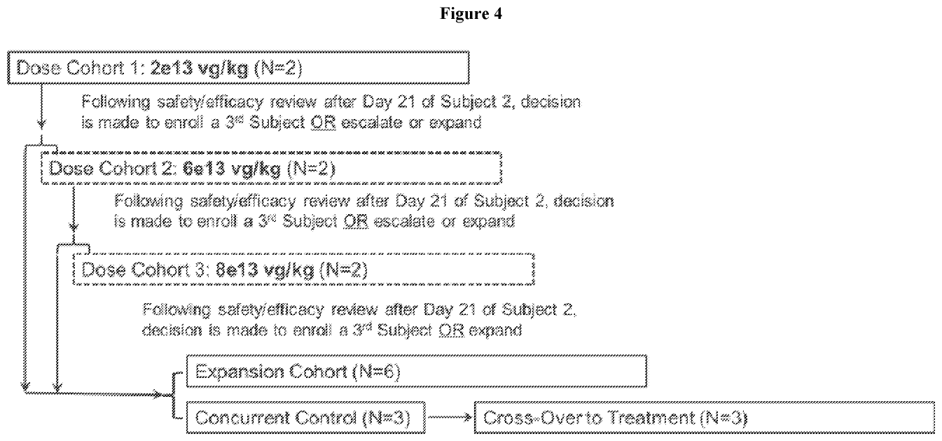

pHMI-hPAH-TC-025 vector packaged in AAVHSC15 capsid will be administered intravenously over approximately 2-4 hours in the clinical research unit setting. The 3 cohorts of dose levels of pHMI-hPAH-TC-025 vector packaged in AAVHSC15 capsid to be investigated in the study are: (1) 2e13 vg/kg; (2) 6e13 vg/kg; (3) 8e13 vg/kg; and (4) 1e14 vg/kg. Upper limit of dosage will be 2e14 vg/kg. FIG. 4 is a study design schematic showing dose cohorts 1 to 3.

One day prior to administration of pHMI-hPAH-TC-025 vector packaged in AAVHSC15 capsid, subjects will be started on prophylactic oral prednisolone therapy which will be administered for 20 weeks as follows:
Prednisolone 60 mg/day×2 weeks
Prednisolone 40 mg/day×6 weeks
Prednisolone 30 mg/day×3 weeks
Prednisolone 20 mg/day×3 weeks
Prednisolone 10 mg/day×5 weeks
Prednisolone 5 mg/day×1 week (the investigator has the discretion to continue the 5 mg/day dose for a total of 2 weeks (i.e., 1 additional week) based on clinical judgment)

If a subject experiences elevated AST and/or ALT>2×ULN during the prophylactic prednisolone regimen, the steroid will be re-escalated or re-started at 60 mg/day, and then tapered again, according to the above schedule.

If a subject experiences elevated AST and/or ALT>2×ULN following the end of the prophylactic prednisolone regimen, the steroid will be re-started according to the following schedule, with modification allowed by the Investigator (in consultation with the Sponsor Medical Monitor or designee) based on laboratory parameters, the subject's medical history and clinical course, and/or subject tolerance of the regimen:
Prednisolone 60 mg/day×2 weeks or until ALT and AST levels have declined to ≤subject's baseline levels
Prednisolone 40 mg/day×2 weeks
Prednisolone 30 mg/day×2 weeks
Prednisolone 20 mg/day×2 weeks
Prednisolone 10 mg/day×2 weeks
Prednisolone 5 mg/day×2 weeks

The prednisolone taper below 60 mg/day should not be started until the ALT and AST have declined to baseline (pre-administration) levels, provided the subject tolerates the regimen. The dose of 60 mg/day may be continued up to 4 weeks, or the dose re-escalated to that level, if the ALT/AST rise again and/or it is otherwise determined to be necessary in the judgment of the Investigator in consultation with the Sponsor's Medical Monitor or designee. After the ALT/AST have reduced again and/or the clinical situation is controlled, the subsequent taper may then proceed. The 60 mg/day dose should not exceed 4 weeks.

Subjects whose transaminase values continue to rise at 60 mg/day or 40 mg/day should be considered for treatment with intravenous methylprednisolone instead of oral prednisolone at the discretion of the Investigator, in consultation with the Sponsor's Medical Monitor or designee.

If the Investigator determines that oral prednisone should be administered instead of oral prednisolone, the Investigator must discuss the rationale with the Sponsor's Medical Monitor or designee and obtain Sponsor's Medical Monitor's or designee's approval to allow this alternate steroid. It is anticipated that the dosing of prednisone and the taper schedule should be the same as that for prednisolone. The rationale, approval, and administration of prednisone instead of prednisolone must be documented in the subject's study record.

If acute illness with fever occurs while the subject is on 5 mg/day or 10 mg/day of prednisolone, the dose should be doubled for 48 hours and then resumed at the previous dose and tapering schedule.

At any time that a subject on steroids (and up to 6 months following steroid discontinuation) undergoes major surgery or experiences major trauma or illness, stress steroids should be administered according to standard of care. Subjects will be informed of the risks of steroids, including HPA axis suppression and other steroid-related side effects.

The intention is that Investigators and subjects will follow the steroid regimens as described in the protocol. However, it is recognized that uncommon situations could arise in which it is in the best interest of the subject to have the steroid dose reduced or discontinued. The steroid regimens are intended to suppress or control the immune response to the gene therapy and thereby preserve gene expression in the hepatocytes. A potential additional goal for the steroid regimen that addresses increased LFTs is to protect the liver cells by addressing the liver inflammation, if severe. These factors need to be taken into account if the Investigator is considering reducing the dose of steroids or discontinuing the steroids in the setting of a clinically important event such as steroid-related psychosis or herpetic corneal ulceration. If the Investigator sees the need to reduce or discontinue the steroids, the Sponsor's Medical Monitor or designee must be consulted prior to the Investigator modifying the steroid dosing (unless time does not permit this in an immediately life-threatening situation).

### Concomitant Therapy

Subjects will continue their usual dietary regimen during the screening period. The baseline diet will be established during the screening period, defined as ±25% of average total protein intake (intact and medical), whether Phe-restricted or unrestricted. The baseline diet will be maintained following administration of pHMI-hPAH-TC-025 vector packaged in AAVHSC15 capsid. A recommendation to modify the diet may be made at the discretion of the Investigator, Site Dietician, and in consultation with the Sponsor Medical Monitor or designee based on the following guidelines:
At 8 weeks, if three Phe values during the first 8 weeks are ≤360 μmol/L
Prior to 8 weeks, if three Phe values during the first 8 weeks (measured at least one week apart) are <120 μmol/L

Subjects taking medications for the treatment of ADHD, depression, anxiety, or other psychiatric disorders at study entry must be on a stable dose for ≥8 weeks prior to administration of pHMI-hPAH-TC-025 vector packaged in AAVHSC15 capsid and must continue with the same dose regimen throughout the study, unless it is determined that changes should be made for medical reasons.

Use of any medications for PKU, including Kuvan®, LNAA, and Palynziq™, is prohibited for the duration of the study unless the plasma Phe concentration is considered to be unsafe for the subject, and it is determined that such treatment is medically necessary following modification of diet.

### Objectives and Endpoints

The primary objective of the study is to evaluate the safety, tolerability, and efficacy of a single dose of pHMI-hPAH-TC-025 vector packaged in AAVHSC15 capsid when administered to subjects with phenylalanine hydroxylase (PAH) deficiency.

The primary safety endpoint is incidence and severity of treatment emergent adverse events (TEAEs) and serious TEAEs. The primary efficacy endpoint is incidence of sustained plasma Phe concentration of ≤360 μmol/L at 24 weeks following administration of pHMI-hPAH-TC-025 vector packaged in AAVHSC15 capsid. Sustained plasma Phe concentration is defined as at least two plasma Phe measurements ≤360 μmol/L between 16 and 24 weeks.

The secondary objectives of the study are to evaluate the effect of pHMI-hPAH-TC-025 vector packaged in AAVHSC15 capsid on plasma phenylalanine (Phe) concentration relative to treatment guidelines for PKU, to assess durability of response, and to characterize the presence of vector and immune response following administration of pHMI-hPAH-TC-025 vector packaged in AAVHSC15 capsid.

The key secondary endpoint is measurement of plasma Phe concentration at 24 weeks post-treatment. Additional secondary endpoints include:
Incidence of achieving a plasma Phe concentration ≤360 μmol/L at each time point during the study
Incidence of achieving a plasma Phe concentration ≤120 μmol/L at each timepoint during the study
Assessment of presence of vector DNA in blood
Assessment of vector shedding in urine, stool, and saliva
Measurement of anti-AAVHSC15 antibodies (IgG and neutralizing), anti-PAH transgene antibody titers, and cytotoxic T-lymphocyte response (ELISPOT)
Safety and tolerability (including incidence of dose-limiting toxicities)

### Results

The above protocol was modified such that Cohort 3 was administered a dose of 1e14 vg/kg of AAV vector, rather than 8e13 vg/kg. Accordingly, pHMI-hPAH-TC-025 vector packaged in AAVHSC15 capsid was administered intravenously to six patients, according to three dosing cohorts (two patients each): (1) low-dose cohort: 2e13 vg/kg; (2) mid-dose cohort: 6e13 vg/kg; and (3) high-dose cohort: 1e14 vg/kg.

Table 4 shows a summary of the baseline characteristics for each patient.

TABLE 4
Patient Baseline Characteristics

| | | | | | | Pre-Existing |
|---|---|---|---|---|---|---|
| Cohort | | | | Baseline | Weeks | Underlying |
| (dose | Patient | | | Phe | Post- | Immune |
| level) | # | Sex | Age | (μmol/L) | dosing | Conditions |
| Cohort 1 | 1 | F | 36 | 1140 | 52 | |
| (low- | | | | | (End of | |
| dose) | | | | | Study) | |
| | 2 | M | 49 | 1020 | 52 | |
| | | | | | (End of | |
| | | | | | Study) | |
| Cohort 2 | 3 | M | 24 | 1010 | 48 | |
| (mid- | 4 | F | 21 | 1060 | 44 | Asthma, Seasonal |
| dose) | | | | | | Allergies |
| Cohort 3 | 5 | F | 31 | 1660 | 28 | Asthma, Eczema, |
| (high- | | | | | | Food Allergies, |
| dose) | | | | | | Environmental |
| | | | | | | Allergies |
| | 6 | M | 33 | 1060 | 13 | |

The majority of patients self-liberalized dietary intact protein, e.g., increased their dietary intake of natural protein, and/or Phe intake. Table 5 shows a summary of change in protein intake for each patient. For each subject, a mean post-baseline value was derived for each nutrient by summing all post-baseline values and dividing by the total number of visits. Mean post-baseline change was then calculated by subtracting the baseline value from the mean post-baseline value for each nutrient. % CFB was derived by dividing the mean post-baseline change value by the baseline value and multiplying by 100 for each nutrient.

TABLE 5
Change in Patient Dietary Protein Intake

| | | Mean % Change From Baseline | | | |
|---|---|---|---|---|---|
| Cohort | | Intact | Total | Phe | Tyr |
| (dose level) | Patient | Protein | Protein | Intake | Intake |
| Cohort 1 | 1 | +14.2 | +4.5 | +0.4 | −10.8 |
| (low-dose) | 2 | +66.0 | −3.9 | +78.5 | −14.7 |
| Cohort 2 | 3 | −30.0 | −4.8 | +100.6 | −1.9 |
| (mid-dose) | 4 | +140.5 | −9.6 | +289.0 | −75.6 |
| Cohort 3 | 5 | −16.9 | −16.9 | −18.5 | −21.0 |
| (high-dose) | 6 | +45.4 | +8.8 | +41.8 | +3.4 |

It was found that pHMI-hPAH-TC-025 vector packaged in AAVHSC15 capsid was generally well tolerated. No treatment-related serious adverse events (SAEs) were reported in the six dosed patients. One non-treatment-related SAE was observed in a patient that developed Herpes zoster.

Transaminitis, as evidenced by elevated ALT, occurred in five patients. Grade 1 and 3 ALT increases were observed in Cohorts 2 and 3, and managed with increased steroids. Two Grade 3 ALT elevations were observed in patients 4 and 5, both with pre-existing immune conditions. Normal cortisol levels were observed in patient 4 during planned high-dose, prophylactic steroid therapy. The severity of ALT increase was found to be associated with pre-existing immune conditions. Without being bound to any theory, ALT increases may impact efficacy of pHMI-hPAH-TC-025 vector packaged in AAVHSC15 capsid. In Cohorts 2 and 3, Phe reductions were found to be greater in patients with Grade 1 ALT elevations compared to patients with Grade 3 ALT elevations (p<0.05; Post-hoc comparison of Patients 3&6 vs Patients 4&5 using repeated measures MANOVA/regression analysis). Table 6 summarizes the ALT elevation status for each patient. ALT Grades are based on Common Terminology Criteria for Adverse Events (CTCAE) Version 5.

TABLE 6
Patient ALT Elevation Status

| Patient # | Peak ALT Grade (times ULN) |
|---|---|
| 1 | WNL |
| 2 | Grade 1 (1.4 × ULN) |
| 3 | Grade 1(1.73 × ULN) |
| 4 | Grade 3 (11.02 × ULN) |
| 5 | Grade 3 (5.1 × ULN) |
| 6 | Grade 1 (3.2 × ULN)* |

*Grade 1 based on baseline ALT value above ULN.

### Example 4: Clinical Study of a PAH Transfer Vector

This example describes a protocol for a Phase 1/2, randomized, concurrently-controlled, dose escalation study to evaluate the safety and efficacy of an rAAV comprising the pHMI-hPAH-TC-025 vector packaged in AAVHSC15 capsid, in adult phenylketonuria (PKU) subjects with PAH deficiency.

Subjects will undergo screening assessments prior to study entry, with the screening period lasting up to 47 days. One day prior to administration of pHMI-hPAH-TC-025 vector packaged in AAVHSC15 capsid, subjects will be admitted to the clinical research unit and prophylactic steroid administration will be initiated. A single administration of pHMI-hPAH-TC-025 vector packaged in AAVHSC15 capsid lasting approximately 2-4 hours will occur in a hospital setting. Subjects may be discharged home after they have been observed for at least 24 hours in the hospital following administration of pHMI-hPAH-TC-025 vector packaged in AAVHSC15 capsid, provided they are clinically stable. Subjects will continue their usual dietary regimen (either Phe-restricted or unrestricted diet) during the screening period and following administration of pHMI-hPAH-TC-025 vector packaged in AAVHSC15 capsid for the duration of the study unless otherwise indicated. Decisions to modify the diet will be made by the investigator for that subject following consultation among sponsor's medical monitor or designee, the investigator, and the site dietician.

Subjects will undergo safety and efficacy observation for 52 weeks in this study following administration of pHMI-hPAH-TC-025 vector packaged in AAVHSC15 capsid.

There are 3 dose levels of pHMI-hPAH-TC-025 vector packaged in AAVHSC15 capsid planned to be evaluated in Part 1 (dose escalation) of the study. In addition, depending on the emerging safety and efficacy profile observed, additional dose cohorts may be added to investigate either intermediate or higher dose levels up to a maximum of 1.5e14 vg/kg. At a given dose level, 2 subjects will be enrolled and dosing staggered with at least a 21-day interval between dosing of each subject. At least 21-day safety follow-up and available plasma Phe concentration data for each of the first 2 subjects at each dose level will be reviewed by sponsor's medical monitor or designee before pHMI-hPAH-TC-025 vector packaged in AAVHSC15 capsid can be administered to the subsequent subject in that cohort.

Additional dose cohorts may be added by the sponsor to investigate intermediate or higher doses, depending on the observed safety and efficacy profile. The maximum dose for the study will not exceed 1.5e14 vg/kg. Any dose escalation or dose expansion, including the decision to initiate treatment in an intermediate or high dose level, will be made by sponsor's medical monitor or designee based on the conclusion that the current emerging safety and plasma Phe response data are supportive and will consider recommendations from a monitoring committee. The data to be reviewed by the monitoring committee includes TEAEs (serious and non-serious), vital signs, physical examinations, ECGs, clinical laboratory tests, and plasma Phe concentration.

Decisions regarding dose escalation and expansion will be based on safety and changes in plasma Phe concentrations relative to the US treatment guidelines for PKU. Treatment guidelines describe the range of 120-360 μmol/L as PAH deficiency not requiring treatment (see, Vockley et al. Genet. Med. (2014) 16(2): 188-200), and the normal range of blood Phe (e.g., plasma Phe) for an individual without hyperphenylalaninemia (HPA) or PKU as 58±14 (SD) mol/L (see, Camp et al. Mol. Genet. Metab. (2014) 112(2):87-122).

In Part 2 dose expansion, evaluation of up to 2 dose levels is planned. Up to 20 additional subjects will be enrolled and randomized in a 2:2:1 fashion to the treatment arms (up to 8 subjects will be randomized at each selected dose level) or to the delayed-treatment control arm (up to 4 subjects). Based on the emerging safety and efficacy data, if a single dose level is selected, randomization will continue in a 2:1 fashion between the treatment and the delayed-treatment control arm. Furthermore, based on supportive safety and efficacy data, additional dose levels may be selected for evaluation in Part 2 and introduced in a 2:1 randomization scheme. There will be no staggered enrollment in Part 2 dose expansion.

### Inclusion and Exclusion Criteria

Suitable subjects for the study will be enrolled according to the inclusion and exclusion criteria set forth in Table 7.

TABLE 7
Inclusion and Exclusion Criteria
Inclusion Criteria:
Diagnosis of PKU due to PAH deficiency.
Two plasma Phe values with a concentration of >600 μmol/L drawn at least 72 hours
apart during the screening period and at least 1 historical value >600 μmol/L in the
24 months prior to screening.
Subject is able to understand the purpose and risks of the study, is willing to provide
informed consent, and is able to comply with all study procedures and 4-year long-
term follow-up.
Adults 18-55 years of age at the time of informed consent.
In the judgment of the site dietitian, subject has the ability and willingness to maintain
their baseline diet ( 25% of average total protein intake [intact and medical]),
whether Phe-restricted or unrestricted, as established during the screening period and
Day −1 (Visit 1) for the treatment arm (receiving pHMI-hPAH-TC-025 vector
packaged in AAVHSC15 capsid) or Week 0 (Visit 1c) for the delayed-treatment
control arm, for the duration of the study, unless otherwise directed.
If applicable, ability to maintain stable dose of medication for attention-
deficit/hyperactivity disorder (ADHD), depression, anxiety, or other psychiatric
disorder for ≥8 weeks prior to enrollment and willing to maintain stable dose
throughout study unless a change is medically indicated.
All females of childbearing potential and sexually active males must be willing to
use highly effective contraception during the study and through 12 months following
administration of pHMI-hPAH-TC-025 vector packaged in AAVHSC15 capsid,
which includes barrier contraception (male or female condom) for 6 months after
administration of pHMI-hPAH-TC-025 vector packaged in AAVHSC15 capsid.
Willing to avoid donation of semen for 12 months following administration of pHMI-
hPAH-TC-025 vector packaged in AAVHSC15 capsid.
Willing to avoid blood, tissue, or organ donation until all vector shedding matrices
are negative.
Exclusion Criteria:
Subjects with PKU that is not due to PAH deficiency.
Presence of anti-AAVHSC15 neutralizing antibodies (at titer >1:5).
History of, or positive test result for, human immunodeficiency virus (HIV) or other
immunosuppressive disorder or medical condition requiring the use of systemic
therapies resulting in immunosuppression or use of systemic immunosuppressive
agents, including corticosteroids, within 30 days prior to administration of pHMI-
hPAH-TC-025 vector packaged in AAVHSC15 capsid.
Subject has known autoimmune condition (exceptions include diabetes, vitiligo,
Graves' disease, Hashimoto's thyroiditis, and psoriasis in the absence of
arthropathy/arthritis).
Current diagnosis of persistent (mild, moderate, or severe) asthma. Mild asthma is
defined as use of short-acting beta agonist >2× per week; moderate asthma is defined
as daily use of short-acting beta agonist.
Subject has current diagnosis of cataracts or glaucoma.
History of hepatitis C virus (HCV) or hepatitis B virus (HBV), current treatment with
an antiviral therapy for HCV or HBV or positive test result for HCV or HBV defined
as:
1. Positive for HCV antibody.
2. Positive test for HBV surface antigen (HBsAg), HBV surface antibody (HBsAb)
and/or HBV core antibody (HBcAb) at screening. Isolated HBsAb positivity with
proof of HBV vaccination with full series is not exclusionary.
Clinically significant liver, biliary, or pancreatic disease by ultrasound at screening
or Grade 3 or Grade 4 fibrosis as assessed by FibroScan at screening or history of
significant underlying liver disease, liver transplant, genetic liver disease, cirrhosis,
nonalcoholic steato-hepatitis (NASH), or other liver condition that would preclude
participation in the study as determined by the investigator.
History of drug abuse or alcoholism that would limit participation in the study, as
determined by the investigator or subjects who exceed moderate drinking levels
defined as: >2 drinks on any single day or >14 drinks per week if male; >1 drink on
any single day or >7 drinks per week if female and/or unwilling to avoid alcohol
intake exceeding moderate drinking levels for the duration of the study.
Liver dysfunction at screening as defined by any of the following:
1. ALT > ULN;
2. AST > ULN;
3. Alkaline phosphatase > ULN;
4. Total bilirubin > ULN;
5. Direct bilirubin > ULN; or
6. International normalized ratio (INR) >1.2
If laboratory assessments fall outside these ranges, repeat testing of the entire liver
panel and prothrombin time/INR is permissible and, if eligibility criteria are met on
retest within the allowable screening period, the subjects may be enrolled after
confirmation with medical monitor and sponsor.
Serum creatinine >1.5× ULN.
Hematology values below the normal range defined by any of the following:
1. Hemoglobin ≤11.0 g/dL for males or ≤10.0 g/dL for females:
2. White blood cells (WBC) ≤3000/μL;
3. Absolute neutrophils ≤1500/μL; or
4. Platelets ≤100,000/μL.
Hemoglobin A1c >6.5% or fasting glucose >126 mg/dL
Any clinically significant abnormal laboratory result at screening, in the opinion of
the investigator.
Contraindication to corticosteroid use or conditions that could worsen in the presence
of corticosteroids, as assessed and determined by the investigator.
Previously received gene therapy for the treatment of any condition.
Subject is pregnant, breastfeeding, or intends to become pregnant during the study
period.
Scheduled or anticipated major surgery during the screening period through 52 weeks
following investigational gene therapy infusion for this study.
Administration of live or live-attenuated vaccination with 30 days prior to screening.
Use of levodopa in the 30 days prior to screening.
Use of any investigational products within 30 days or five half-lives, whichever is
longer, prior to screening.
Current enrollment in any other investigational study.
Presence of an untreated or inadequately treated active infection or an infection
requiring systemic antiviral or antimicrobial therapy at any time during the screening
period, including active tuberculosis (TB) or untreated latent TB infection (LTBI),
determined by positive QuantiFERON test at screening.
1. Testing may be repeated once for an indeterminate QuantiFERON test and result
will be considered positive if test is positive or indeterminate.
2. Positive QuantiFERON testing is considered exclusionary without
documentation of LTBI chemoprophylaxis completion prior to screening.
Use of any medication that is intended to treat PKU as follows:
1. Last dose of PALYNZIQ ™ or LNAA supplements (other than part of fortified
medical food) must be at least 30 days prior to screening laboratory assessment of
plasma Phe to determine eligibility.
2. Last dose of Kuvan ® must be at least 7 days prior to screening laboratory
assessment of plasma Phe to determine eligibility.
Body mass index (BMI) ≥35 kg/m².
Weight >100 kg.
Unable or unwilling to provide informed consent.
Have a clinically significant medical condition that in the investigator's opinion
would pose an unnecessary risk (including history of chronic infection such as HIV
or other chronic diseases), limit the participation of the subject in the study or impact
the ability to interpret study results.
Has a malignancy, or a history of malignancy, with the exception of successfully
treated basal or squamous cell carcinoma of the skin.
Any other condition that would not allow the potential subject to complete follow-up
examinations during the course of the study or, in the opinion of the investigator,
makes the potential subject unsuitable for the study.
If participation in the study is not in the subject's best interest, in the opinion of the
investigator.

### Investigational Product, Dosage, and Mode of Administration

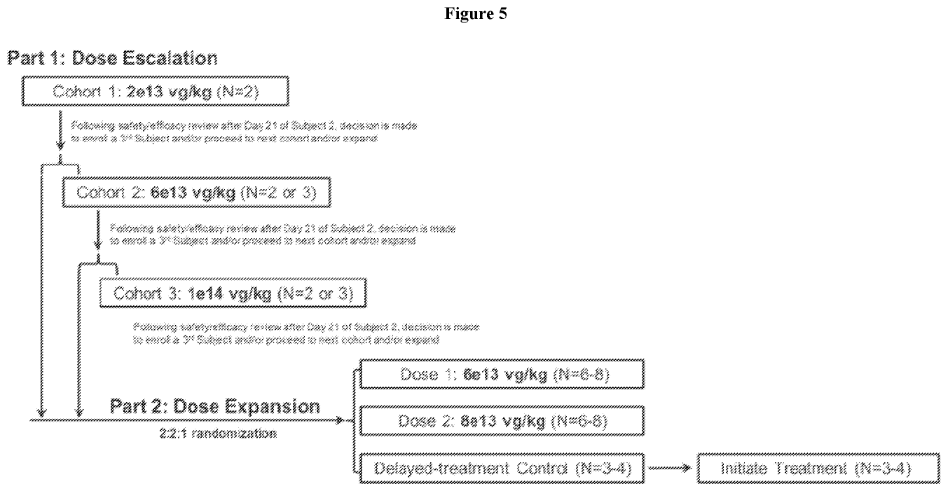

pHMI-hPAH-TC-025 vector packaged in AAVHSC15 capsid will be administered intravenously over approximately 2-4 hours. The 3 dose levels of pHMI-hPAH-TC-025 vector packaged in AAVHSC15 capsid planned to be investigated in the study are (FIG. 5 is a study design schematic showing dose cohorts 1 to 3):
Cohort 1: 2e13 vg/kg
Cohort 2: 6e13 vg/kg
Cohort 3: 1e14 vg/kg

### Prophylactic Corticosteroid Regimen

One day prior to administration of pHMI-hPAH-TC-025 vector packaged in AAVHSC15 capsid, subjects will be started on prophylactic oral dexamethasone therapy which, provided there are no tolerability issues, will be administered for a planned 20 weeks, as follows:
Step 1: Dexamethasone 0.15 mg/kg/day×8 weeks
Step 2: Dexamethasone 0.1 mg/kg/day×4 weeks
Step 3: Dexamethasone 0.075 mg/kg/day×3 weeks
Step 4: Dexamethasone 0.05 mg/kg/day×2 weeks
Step 5: Dexamethasone 0.025 mg/kg/day×2 weeks
Step 6: Dexamethasone 0.0125 mg/kg/day×1 week

Provided there are no tolerability issues, after the initial 8 weeks of dexamethasone treatment, dexamethasone taper below 0.15 mg/kg/day can be started if the ALT and AST are within normal range. The investigator has the discretion to consider slower taper at Step 5 or 6 corticosteroid dosing based on clinical judgment but should consider switching to prednisolone 10 mg/day (Step 5) after 2 weeks or 5 mg/day (Step 6) after 1-2 weeks.

### Corticosteroid Treatment in Response to Elevated Liver Function Tests

If a subject experiences elevated AST and/or ALT>1.5×ULN during the prophylactic dexamethasone regimen, the corticosteroid will be re-escalated or re-started at 0.15 mg/kg/day, and then tapered again, according to the schedule described in Section 11.4.1. In addition, LFTs will be monitored at least 2 times per week until the abnormality has resolved. At discretion of the investigator, the frequency of ALT and AST monitoring may be decreased after stabilization or results are within normal range in consultation with sponsor's medical monitor or designee.

If a subject experiences elevated AST and/or ALT>1.5×ULN following the end of the prophylactic dexamethasone regimen, the corticosteroid will be re-started according to the following schedule, with modification allowed by the investigator (in consultation with sponsor's medical monitor or designee) based on laboratory parameters, the subject's medical history and clinical course, and/or subject tolerance of the regimen:
Step 1: dexamethasone 0.15 mg/kg/day×2 weeks
Step 2: dexamethasone 0.1 mg/kg/day×2 weeks
Step 3: dexamethasone 0.075 mg/kg/day×2 weeks
Step 4: dexamethasone 0.05 mg/kg/day×2 weeks
Step 5: dexamethasone 0.025 mg/kg/day×2 weeks
Step 6: dexamethasone 0.0125 mg/kg/day×2 weeks

Provided there are no tolerability issues, after completion of two weeks of dexamethasone therapy at Step 1, the dexamethasone taper below 0.15 mg/kg/day can be started if the ALT and AST levels are within normal range.

The LFTs will be monitored at least 2 times per week until the abnormality has resolved. The Step 6 dose of dexamethasone (i.e., 0.15 mg/kg/day) may be continued beyond the planned duration of initial treatment, or the dose re-escalated to that level, if ALT/AST levels rise again and/or it is otherwise determined to be necessary in the judgment of the investigator in consultation with sponsor's medical monitor or designee. After ALT/AST levels have decreased again and/or the clinical situation is controlled, the subsequent taper may then proceed. The investigator has discretion to consider other formulations during treatment and to taper corticosteroids more slowly and/or consider intermediate doses or other formulations in consultation with the sponsor. Treatment and tapering of the dexamethasone, prednisolone, or other formulations, should occur under the guidance of an endocrinologist.

Subjects whose transaminase values continue to rise at Step 1 or Step 2 dexamethasone dosing (i.e., 0.15 mg/kg/day or 0.1 mg/kg/day) should be considered, as deemed necessary by the investigator in consultation with sponsor's medical monitor or designee, for treatment preferably with dexamethasone 0.3 mg/kg/day or other corticosteroid formulations or immunosuppressive therapies. Consultation with an endocrinologist should be considered during treatment or taper with dexamethasone or alternate corticosteroid or immunosuppressant regimens.

### Additional Considerations on Corticosteroid Use

If the investigator determines that oral prednisone or prednisolone should be administered instead of oral dexamethasone for Steps 1-6, at a starting dose of approximately 1 mg/kg per day, the investigator must discuss the rationale with sponsor's medical monitor or designee and obtain sponsor's medical monitor's or designee's approval to allow this alternate corticosteroid. The rationale, approval, and administration of prednisone or prednisolone, or other formulations, instead of dexamethasone must be documented in the subject's study record. Based on emerging efficacy and safety data, a change to prednisolone, prednisone, or other formulations for Steps 1-6 for prophylactic or reactive corticosteroid therapy will be undertaken with a starting dose of approximately 1 mg/kg per day prednisone equivalents and corresponding tapering.

The intention is that investigators and subjects will follow the corticosteroid regimens as described in the protocol. However, it is recognized that uncommon situations could arise in which it is in the subject's best interest to have the corticosteroid dose reduced or discontinued. The corticosteroid regimens are intended to suppress or control the immune response to the gene therapy and thereby preserve gene expression in the hepatocytes. A potential additional goal for the corticosteroid regimen that addresses increased LFTs is to protect the liver cells by addressing the liver inflammation, if severe. These factors need to be taken into account if the investigator is considering reducing the dose of corticosteroids or discontinuing them in the setting of a clinically important event, such as corticosteroid-related psychosis or herpetic corneal ulceration. If the investigator deems it necessary to reduce or discontinue the corticosteroids, sponsor's medical monitor or designee must be consulted prior to the investigator modifying the corticosteroid dosing (unless time does not permit this in an immediately life-threatening situation). Consultation with an endocrinologist should be considered during treatment or taper with dexamethasone or alternate corticosteroid regimens.

### Stress Corticosteroids

If acute illness with fever occurs while the subject is on 0.025 mg/kg/day or lower of dexamethasone (i.e., Step 5 or 6), or prednisone or prednisolone equivalent dose level, the dose should be doubled for 48 hours and then resumed at the previous dose and tapering schedule.

At any time that a subject on corticosteroids (and up to 6 months following corticosteroid discontinuation) undergoes major surgery or experiences major trauma or illness, stress corticosteroids should be administered according to standard of care. Subjects will be informed of the risks of corticosteroids, including HPA axis suppression and other corticosteroid-related side effects.

### Vaccinations—Herpes Zoster

Subjects with planned immunosuppression are recommended per CDC guidelines to receive vaccination with SHINGRIX® (Zoster vaccine Recombinant, Adjuvanted) consisting of 2 doses administered 2-6 months apart unless contraindicated.

The vaccine series need not be restarted if more than 6 months have elapsed since the first dose; however, the efficacy of alternative dosing regimens has not been evaluated, data regarding the safety of alternative regimens are limited, and individuals might remain at risk for herpes zoster during a longer than recommended interval between doses 1 and 2.

For subjects without (or uncertain) history of previous vaccination, the first dose of SHINGRIX® should ideally be administered at least 6 weeks prior to screening, and not more than 6 months prior to screening, unless contraindicated or subject declines vaccination. The second dose of SHINGRIX® should then be administered during screening and at least 14 days prior to administration of pHMI-hPAH-TC-025 vector packaged in AAVHSC15 capsid for subjects randomized to the treatment arm, unless contraindicated or subjects declines vaccination. If this is not feasible, the first dose of SHINGRIX® will be administered during screening for both the treatment and the delayed-treatment control arms, and at least 14 days prior to administration of pHMI-hPAH-TC-025 vector packaged in AAVHSC15 capsid for subjects randomized to the treatment arm, unless contraindicated or subjects declines vaccination. For the treatment arm, the second dose of SHINGRIX® will then be administered at Week 24, or 4 weeks after discontinuation of corticosteroids, whichever is later unless acutely ill and then will be administered at a subsequent visit. For the delayed-treatment control arm, the second dose of SHINGRIX® will then be administered at Week 8, unless acutely ill and then will be administered at a subsequent visit. If this occurs, vaccination will need to occur at least 3 weeks prior to receipt of pHMI-hPAH-TC-025 vector packaged in AAVHSC15 capsid.

Persons known to be VZV negative: Screening for a history of varicella (either verbally or via laboratory serology) before vaccination for herpes zoster is not recommended. However, in persons known to be VZV negative via serologic testing, ACIP guidelines for varicella vaccination should be followed. RZV has not been evaluated in persons who are VZV seronegative and the vaccine is not indicated for the prevention of chickenpox (varicella).

Vaccinations Streptococcus pneumoniae Vaccination (PCV13 and PPSV23)

Subjects with planned immunosuppression are recommended per CDC guidelines to receive both PCV13 and PPSV23, with PPSV23 administered at least 8 weeks after PCV13. For subjects without (or uncertain) history of previous vaccination, PCV13 will be administered ideally at least 6 weeks prior to screening and PPSV23 will administered during screening and up to 14 days prior to administration of pHMI-hPAH-TC-025 vector packaged in AAVHSC15 capsid unless contraindicated or subject declines vaccination. However, if this is not feasible, unless contraindicated or subjects declines vaccination, PCV13 will be administered during screening and up to 14 days prior to administration of pHMI-hPAH-TC-025 vector packaged in AAVHSC15 capsid on and PPSV23 will be administered at Week 8 unless acutely ill and then PPSV23 will be given at a subsequent visit. As reduced immunogenicity has been reported during period of immunosuppression, for subjects randomized to the treatment arm, antibody titers against PPSV23 serotypes will be assessed during Week 24, or at least 8 weeks after PPSV23 administration and at least 4 weeks after discontinuation of corticosteroids, whichever is later, to ensure a normal or adequate response. A protective response is defined as a titer ≥1.3 μg/ml for at least 70% of the serotypes. Re-administration of PPSV23 should occur in the absence of protective immunity.

### Vaccinations—Influenza

Inactivated influenza vaccination for the current flu season will be administered during screening at least 2 weeks prior to administration of pHMI-hPAH-TC-025 vector packaged in AAVHSC15 capsid for subjects without documentation of receipt unless contraindicated. If not indicated at the time of screening, or vaccination for the current season is unavailable, subjects will receive vaccination as seasonally appropriate during the study.

### Concomitant Therapy

Subjects will continue their usual dietary regimen during the screening period. The baseline diet will be documented by the site dietitian for each subject during the screening period and Baseline Visit (Visit 1 Day −1) for the treatment arm (receiving pHMI-hPAH-TC-025 vector packaged in AAVHSC15 capsid) or Week 0 (Visit 1c) for the delayed-treatment control arm. Subjects should maintain a consistent diet, which is defined as ±25% of baseline average total protein intake (intact and medical), whether Phe-restricted or unrestricted for the duration of the study, unless instructed by the site investigator or dietitian to support normal health and nutrition or to manage low plasma Phe concentration (i.e., <30 μmol/L).

Additionally, a recommendation to modify the diet following administration of pHMI-hPAH-TC-025 vector packaged in AAVHSC15 capsid may be made based on achievement of a target Phe concentration, and following consultation among sponsor's medical monitor or designee, the investigator, and the site dietitian. Diet modification will be considered after Week 28 post-administration of pHMI-hPAH-TC-025 vector packaged in AAVHSC15 capsid if the 2 most recent consecutive plasma Phe values (at least 1 week apart) are ≤360 μmol/L.

Subjects taking medications for the treatment of ADHD, depression, anxiety, or other psychiatric disorders at study entry must be on a stable dose for ≥8 weeks prior to administration of pHMI-hPAH-TC-025 vector packaged in AAVHSC15 capsid and must continue with the same dose regimen throughout the study, unless it is determined that changes should be made for medical reasons.

Use of any medications for PKU, including Kuvan®, LNAA supplements (other than part of fortified medical food), and PALYNZIQ™, is prohibited for the duration of the study unless the plasma Phe concentration is considered to be unsafe for the subject in the opinion of the investigator, and the investigator, in consultation with sponsor's medical monitor or designee, determines that such treatment is medically necessary following modification of diet.

### Objectives and Endpoints

The objectives and endpoints of this clinical study are outlined in Table 8.

TABLE 8
Study Objectives and Endpoints

| | |
|---|---|
| Objectives | Endpoints |

Part 1 (dose selection)
Primary

| | |
|---|---|
| Determine the safety of a single | Incidence and severity of treatment- |
| administration of pHMI-hPAH-TC-025 | emergent adverse events (TEAEs) |
| vector packaged in AAVHSC15 capsid | Change from baseline in clinical laboratory |
| | testing (serum chemistry, including liver |
| | function tests, hematology, and urinalysis) |
| | Change from baseline in 12-lead |
| | electrocardiograms (ECGs), vital signs, |
| | physical examinations |
| Determine an efficacious dose for single | Change from baseline in mean plasma Phe |
| administration of pHMI-hPAH-TC-025 | levels within each dose cohort during Weeks |
| vector packaged in AAVHSC15 capsid | 24-28 post-administration of pHMI-hPAH- |
| | TC-025 vector packaged in AAVHSC15 |
| | capsid |
| | Incidence of plasma Phe concentration of |
| | ≤360 μmol/L within each dose cohort by |
| | Week 28 post-administration of pHMI- |
| | hPAH-TC-025 vector packaged in |
| | AAVHSC15 capsid |

Part 2 (dose expansion)
Primary

| | |
|---|---|
| To evaluate the effect of pHMI-hPAH-TC- | Change from baseline in mean plasma Phe |
| 025 vector packaged in AAVHSC15 capsid | levels during Weeks 24-28 post- |
| on plasma Phe concentration following a | administration of pHMI-hPAH-TC-025 |
| single administration of pHMI-hPAH-TC- | vector packaged in AAVHSC15 capsid |
| 025 vector packaged in AAVHSC15 capsid | |

Secondary

| | |
|---|---|
| To evaluate the effect of pHMI-hPAH-TC- | Incidence plasma Phe concentration |
| 025 vector packaged in AAVHSC15 capsid | thresholds (i.e., ≤600 μmol/; ≤360 μmol/L; to |
| on plasma Phe concentration relative to | ≤120 μmol/L) up to Week 28 post |
| treatment guidelines for PKU | administration of pHMI-hPAH-TC-025 |
| | vector packaged in AAVHSC15 capsid |
| | Incidence of plasma Phe concentration |
| | thresholds (i.e., ≤600 μmol/; ≤360 μmol/L; to |
| | ≤120 μmol/L) up to Week 52 post |
| | administration of pHMI-hPAH-TC-025 |
| | vector packaged in AAVHSC15 capsid |
| | Incidence of sustained plasma Phe |
| | concentration thresholds (i.e., ≤600 μmol/L; |
| | ≤360 μmol/L; ≤120 μmol/L) through Week |
| | 52 following administration of HMI 102 |
| Assess durability of response | Incidence of achieving mean plasma Phe |
| | ≤360 μmol/L 48-52 weeks post- |
| | administration of pHMI-hPAH-TC-025 |
| | vector packaged in AAVHSC15 capsid |
| | Incidence of achieving mean plasma Phe |
| | ≤120 μmol/L 48-52 weeks post- |
| | administration of pHMI-hPAH-TC-025 |
| | vector packaged in AAVHSC15 capsid |
| | Incidence of achieving mean plasma Phe |
| | ≤360 μmol/L 48-52 weeks in patients who |
| | are responders prior to Week 28 post- |
| | administration of pHMI-hPAH-TC-025 |
| | vector packaged in AAVHSC15 capsid |
| | Change from baseline in mean plasma Phe |
| | levels during Weeks 48-52 post- |
| | administration of pHMI-hPAH-TC-025 |
| | vector packaged in AAVHSC15 capsid |
| Assess the changes in dietary protein intake | Change from baseline in natural protein |
| | intake at Week 52 post-administration of |
| | pHMI-hPAH-TC-025 vector packaged in |
| | AAVHSC15 capsid |
| | Change from baseline in total protein intake |
| | at Week 52 post-administration of pHMI- |
| | hPAH-TC-025 vector packaged in |
| | AAVHSC15 capsid |
| | Incidence of >80% Daily Recommended |
| | Intake (DRI) protein at Week 52 post- |
| | administration of pHMI-hPAH-TC-025 |
| | vector packaged in AAVHSC15 capsid |
| | Change from baseline in Phe intake |
| | (Phe/total protein vs absolute Phe intake) |
| | during Weeks 48-52 post-administration of |
| | pHMI-hPAH-TC-025 vector packaged in |
| | AAVHSC15 capsid |
| | Incidence of discontinuing medical food |
| | intake at Week 52 post-administration of |
| | pHMI-hPAH-TC-025 vector packaged in |
| | AAVHSC15 capsid |
| Assess the safety of a single administration | Incidence and severity of TEAEs |
| of pHMI-hPAH-TC-025 vector packaged in | Assessment of presence of vector DNA in |
| AAVHSC15 capsid | blood |
| | Assessment of vector shedding in urine, |
| | stool, and saliva |
| | Measurement of anti-AAVHSC15 |
| | antibodies (IgG and neutralizing), anti-PAH |
| | antibody titers, and recall antigen specific |
| | IFN gamma T-cell evaluation (interferon |
| | gamma enzyme-linked immunospot |
| | [ELISPOT]) |
| | Change from baseline in clinical laboratory |
| | testing (serum chemistry including liver |
| | function tests, hematology, and urinalysis) |
| | Incidence and severity of ABS Is |
| | Change from baseline in 12-lead |
| | electrocardiograms (ECGs), vital signs, |
| | physical examinations |
| Assess changes in measures in executive | Changes in neuropsychiatric outcomes |
| function following a single administration of | following administration of pHMI-hPAH- |
| pHMI-hPAH-TC-025 vector packaged in | TC-025 vector packaged in AAVHSC15 |
| AAVHSC15 capsid | capsid, as assessed by NIR Toolbox |
| | Measures-Cognitive Domain at Weeks 28 |
| | and 52 |
| Explore impact of quality of life following a | Change from baseline in CGI assessment at |
| single administration of pHMI-hPAH-TC- | Week 28 and Week 52 |
| 025 vector packaged in AAVHSC15 capsid | Change from baseline in PGI assessment at |
| | Week 28 and Week 52 |

Exploratory
The exploratory objectives of the Part 2 dose-expansion portion of the study are to evaluate
additional effects of pHMI-hPAH-TC-025 vector packaged in AAVHSC15 capsid on
subjects with PAH deficiency. These additional assessments will be summarized over time
for exploratory analyses.
Summary statistics for plasma Phe concentration at each time point during the study
Summary statistics for plasma Tyrosine concentration at each time point during the study
Summary statistics for plasma Phe/Tyr ratios at each time point during the study
Mean time to plasma Phe concentration ≤120, ≤360, and ≤600 μmol/L
Incidence of a Phe-restricted diet following administration of pHMI-hPAH-TC-025 vector
packaged in AAVHSC15 capsid
Change from baseline in Quality of life (QOL) using the PKU-QOL Questionnaire and
additional QOL measures of PROMIS-Global Health and Neuro-QOL-Cognitive Function
over time
Change from baseline in CGI/PGI over time
Time to response in CGI/PGI assessments over time
Summary statistics for diet diary data over time

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method for reducing the risk of occurrence and/or the severity of a pathogenic disease in a subject receiving a gene therapy and an immunosuppressant regimen, the method comprising: administering a prophylactic vaccine to the subject that will receive the gene therapy and the immunosuppressant regimen.

2. The method of claim 1, wherein the pathogenic disease is herpes zoster and the method comprises: administering a herpes zoster vaccine to the subject that will receive the gene therapy and the immunosuppressant regimen, wherein an initial dose of the herpes zoster vaccine is administered to the subject prior to administration of the immunosuppressant regimen,

3. The method of claim 2, wherein the vaccine:
a) comprises a polysaccharide, a polypeptide, or a nucleic acid;
b) is a subunit vaccine; and/or
c) comprises a varicella zoster virus glycoprotein E antigen, wherein the vaccine comprises one or more of:
(i) a recombinant varicella zoster virus glycoprotein E antigen and/or
(ii) recombinant varicella zoster virus glycoprotein E antigen, monophosphoryl lipid A, and QS-21.

4.-7. (canceled)

8. The method of claim 2, wherein:
a) the initial dose of the vaccine is administered to the subject at least about 6 weeks prior to commencement of the immunosuppressant regimen, optionally wherein at least one subsequent dose of the vaccine is administered to the subject after administration of the initial dose, optionally at least about 2 weeks prior to commencement of the immunosuppressant regimen; and/or
b) the gene therapy is administered to the subject at least 1 day after commencement of the immunosuppressant regimen.

9.-11. (canceled)

12. The method of claim 1, wherein the pathogenic disease is an S. pneumoniae related disease or disorder and the method comprises: administering an S. pneumoniae vaccine to a subject that will receive the gene therapy and the immunosuppressant regimen, wherein an initial dose of the vaccine is administered to the subject prior to administration of the immunosuppressant regimen, optionally wherein the S. pneumoniae related disease or disorder is selected from the group consisting of pneumonia, meningitis, sepsis, and any combination thereof.

13. (canceled)

14. The method of claim 12, wherein the vaccine:
a) comprises a polysaccharide, a polypeptide, or a nucleic acid;
b) is selected from the group consisting of: a live-attenuated vaccine; an inactivated vaccine; a subunit vaccine; a toxoid vaccine; and a nucleic acid vaccine; or
c) is a subunit vaccine selected from the group consisting of a polysaccharide vaccine, a conjugate vaccine, a toxoid vaccine, and a recombinant protein vaccine, optionally wherein the vaccine is a conjugate vaccine, optionally comprising:
(i) purified capsular polysaccharides of one or more of serotypes 1, 3, 4, 5, 6A, 6B, 7F, 9V, 14, 18C, 19A, 19F, and 23F of S. pneumoniae conjugated to CRM197; or
(ii) purified capsular polysaccharides of serotypes 1, 3, 4, 5, 6A, 6B, 7F, 9V, 14, 18C, 19A, 19F, and 23F of S. pneumoniae conjugated to CRM197 (PCV13).

15.-19. (canceled)

20. The method of claim 14, wherein the vaccine is a polysaccharide vaccine, optionally comprising:
a) purified capsular polysaccharides of one or more of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae; or
b) purified capsular polysaccharides of serotypes 1, 2, 3, 4, 5, 6B, 7F, 8, 9N, 9V, 10A, 11A, 12F, 14, 15B, 17F, 18C, 19F, 19A, 20, 22F, 23F, and 33F of S. pneumoniae (PPSV23).

21.-22. (canceled)

23. The method of claim 14, wherein the initial dose of the vaccine comprises PCV13, optionally wherein:
a) the initial dose of the vaccine is administered to the subject at least about 10 weeks prior to commencement of the immunosuppressant regimen;
b) at least one subsequent dose of the vaccine is administered to the subject at least about 8 weeks after administration of the initial dose, and optionally the at least one subsequent dose of the vaccine:
(i) comprises PPSV23; and/or
(ii) is administered to the subject at least about 2 weeks prior to commencement of the immunosuppressant regimen; and/or
c) the gene therapy is administered to the subject at least 1 day after commencement of the immunosuppressant regimen.

24.-28. (canceled)

29. The method of claim 1, wherein the pathogenic disease is influenza and the method comprises administering an influenza vaccine to a subject that will receive or has received the gene therapy and the immunosuppressant regimen, optionally, wherein the vaccine:
a) comprises a polysaccharide, a polypeptide, or a nucleic acid;
b) is selected from the group consisting of: a live-attenuated vaccine; an inactivated vaccine; a subunit vaccine; a toxoid vaccine; and a nucleic acid vaccine, optionally wherein
(i) the vaccine is a subunit vaccine selected from the group consisting of a polysaccharide vaccine, a conjugate vaccine, a toxoid vaccine, and a recombinant protein vaccine;
(ii) the vaccine is a nucleic acid vaccine selected from the group consisting of a DNA-based vaccine, an RNA-based vaccine, and a recombinant vector vaccine;
and wherein:
c) the vaccine is administered to the subject:
(i) prior to commencement of the immunosuppressant regimen;
(ii) at least about two weeks prior to commencement of the immunosuppressant regimen; and/or
(iii) after commencement of the immunosuppressant regimen;
d) the gene therapy is administered to the subject at least 1 day after commencement of the immunosuppressant regimen, and optionally comprises a recombinant viral vector, optionally wherein the recombinant viral vector comprises a transgene, optionally wherein the transgene encodes a polypeptide; and/or wherein the transgene encodes an miRNA, shRNA, siRNA, antisense RNA, gRNA, antagomir, miRNA sponge, RNA aptazyme, RNA aptamer, lncRNA, ribozyme, or mRNA; and/or
e) the immunosuppressant regimen comprises administration of a glucocorticoid selected from the group consisting of hydrocortisone, cortisone acetate, prednisone, prednisolone, methylprednisone, triamcinolone, dexamethasone, and betamethasone.

30-42. (canceled)

43. The method of claim 29, wherein:
a) the polypeptide is selected from the group consisting of β-globin, hemoglobin, tissue plasminogen activator, and coagulation factors; colony stimulating factors (CSF);
b) the polypeptide is an interleukin, optionally wherein the interleukin is selected from the group consisting of IL-1, IL-2, TL-3, TL-4, TL-5, TL-6, TL-7, IL-8, and TL-9;
c) the polypeptide is a growth factor, optionally wherein the growth factor is selected from the group consisting of a keratinocyte growth factor (KGF), stem cell factor (SCF), fibroblast growth factor (FGF), basic FGF, acidic FGF, hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), a neurotrophin, platelet-derived growth factor (PDGF), thrombopoietin (TPO), transforming growth factor alpha (TGF-a), and transforming growth factor beta (TGF-0);
d) the polypeptide is a soluble receptor, optionally wherein the soluble receptor is selected from the group consisting of a soluble TNF-a receptor, a soluble interleukin receptor, a soluble γ/Δ T cell receptor, and ligand-binding fragments of a soluble receptor;
the polypeptide is an enzyme, optionally wherein the enzyme is selected from the group consisting of a-glucosidase, imiglucerase, β-glucocerebrosidase, and alglucerase;
e) the polypeptide is an enzyme activator, optionally wherein the enzyme activator is tissue plasminogen activator;
the polypeptide is a chemokine, optionally wherein the chemokine is selected from the group consisting of IP-10, monokine induced by interferon-gamma (Mig), Groα/IL-8, RANTES, MIP-1a, MIP-1β, MCP-1, and PF-4;
f) the polypeptide is an angiogenic agent, optionally wherein the angiogenic agent is VEGF, VEGF121, VEGF165, VEGF-C, VEGF-2, glioma-derived growth factor, angiogenin, and angiogenin-2;
g) the polypeptide is an anti-angiogenic agent, optionally wherein the anti-angiogenic agent is selected from the group consisting of a soluble VEGF receptor;
h) the polypeptide is a protein vaccine;
i) the polypeptide is a neuroactive peptide, optionally wherein the neuroactive peptide is selected from the group consisting of a nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, a glucagon, vasopressin, angiotensin II, thyrotropin-releasing hormone, vasoactive intestinal peptide, and a sleep peptide;
j) the polypeptide is selected from the group consisting of a thrombolytic agent, atrial natriuretic peptide, relaxin, glial fibrillary acidic protein, follicle stimulating hormone (FSH), human alpha-1 antitrypsin, leukemia inhibitory factor (LIF), a tissue factor, a macrophage activating factor, a tumor necrosis factor (TNF), neutrophil chemotactic factor (NCF), a tissue inhibitor of a metalloproteinase, vasoactive intestinal peptide, angiogenin, angiotrophin, fibrin, hirudin, an IL-1 receptor antagonist, ciliary neurotrophic factor (CNTF), brain-derived neurotrophic factor (BDNF), neurotrophin 3, neurotrophin 4/5, glial cell derived neurotrophic factor (GDNF), aromatic amino acid decarboxylase (AADC), Factor VIII, Factor IX, Factor X, dystrophin, mini-dystrophin, lysosomal acid lipase, and phenylalanine hydroxylase (PAH);
k) the polypeptide is a glycogen storage disease-related enzyme, optionally wherein the glycogen storage disease-related enzyme is selected from the group consisting of glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, and glycogen synthase;
l) the polypeptide is a lysosomal enzyme, optionally wherein the lysosomal enzyme is selected from the group consisting of iduronate-2-sulfatase (I2S), and arylsulfatase A;
the polypeptide is a mitochondrial protein, optionally wherein the mitochondrial protein is frataxin (FXN);
m) the polypeptide is a protein that may be defective in one or more lysosomal storage diseases, wherein the protein is selected from the group consisting of α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, capthepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN6, CLN8, LYST, MYOV, RAB27A, melanophilin, and AP3 β-subunit;
n) the polypeptide is an antibody or fragment thereof, optionally wherein the antibody is selected from the group consisting of muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab;
o) the polypeptide is a nuclease, optionally wherein the nuclease is selected from the group consisting of a zinc finger nuclease (ZFN), a transcription activator-like effector nuclease (TALEN), a homing endonuclease, and a meganuclease;
p) the polypeptide is an RNA-guided nuclease, wherein the RNA-guided nuclease is selected from the group consisting of a Cas3, Cas8a, Cas5, Cas8b, Cas8c, Cas10d, Cse1, Cse2, Csy1, Csy2, Csy3, GSU0054, Cas10, Csm2, Cmr5, Csx10, Csx11, Csx10, Csf1, Cas9, Csn2, Cas4, Cpf1, C2c1, C2c3, Cas13a, Cas13b, Cas13c, and Cas12a/Cpf1; or
q) the polypeptide is a reporter, optionally wherein the reporter is selected from the group consisting of β-lactamase, β-galactosidase (LacZ), alkaline phosphatase, thymidine kinase, green fluorescent protein (GFP), red fluorescent protein (RFP), chloramphenicol acetyltransferase (CAT), luciferase, CD2, CD4, CD8, the influenza hemagglutinin protein, and Myc.

44. (canceled)

45. The method of claim 43, wherein the recombinant viral vector:
a) is selected from the group consisting of a retroviral vector, a lentiviral vector, an adenoviral vector, and an adeno-associated virus vector;
b) is an adeno-associated virus (AAV) vector, optionally wherein the AAV comprises
(i) a recombinant AAV genome; and
(ii) an AAV capsid comprising an AAV capsid protein, optionally wherein the capsid protein:
(a) comprises the amino acid sequence of amino acids 1-736 of SEQ ID NO: 1, 2, 3, 4, 5, 6, 7, 8, 9, 10, 11, 12, 13, 15, 16, or 17;
(b) is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PHP.S; and/or
(c) comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17; and/or
(d) comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17, optionally wherein
i. the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
ii. the amino acid in the capsid protein corresponding to amino acid 626 of SEQ ID NO: 16 is G, and the amino acid in the capsid protein corresponding to amino acid 718 of SEQ ID NO: 16 is G;
iii. the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
iv. the amino acid in the capsid protein corresponding to amino acid 505 of SEQ ID NO: 16 is R, and the amino acid in the capsid protein corresponding to amino acid 687 of SEQ ID NO: 16 is R;
v. the amino acid in the capsid protein corresponding to amino acid 346 of SEQ ID NO: 16 is A, and the amino acid in the capsid protein corresponding to amino acid 505 of SEQ ID NO: 16 is R; or
vi. the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

46.-51. (canceled)

52. The method of claim 45, wherein the AAV capsid protein comprises:
a) the amino acid sequence of amino acids 203-736 of SEQ ID NO: 1, 2, 3, 4, 5, 6, 7, 8, 9, 10, 11, 12, 13, 15, 16, or 17;
b) an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17, optionally wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G; optionally wherein:
(i) the amino acid in the capsid protein corresponding to amino acid 626 of SEQ ID NO: 16 is G, and the amino acid in the capsid protein corresponding to amino acid 718 of SEQ ID NO: 16 is G;
(ii) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(iii) the amino acid in the capsid protein corresponding to amino acid 505 of SEQ ID NO: 16 is R, and the amino acid in the capsid protein corresponding to amino acid 687 of SEQ ID NO: 16 is R;
(iv) the amino acid in the capsid protein corresponding to amino acid 346 of SEQ ID NO: 16 is A, and the amino acid in the capsid protein corresponding to amino acid 505 of SEQ ID NO: 16 is R; or
(v) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

53-55. (canceled)

56. The method of claim 45, wherein the AAV capsid protein:
a) comprises the amino acid sequence of amino acids 138-736 of SEQ ID NO: 1, 2, 3, 4, 5, 6, 7, 9, 10, 11, 12, 13, 15, 16, or 17;
b) comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17, optionally wherein:
(i) the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G;
(ii) the amino acid in the capsid protein corresponding to amino acid 2 of SEQ ID NO: 16 is T, and the amino acid in the capsid protein corresponding to amino acid 312 of SEQ ID NO: 16 is Q;
(iii) the amino acid in the capsid protein corresponding to amino acid 65 of SEQ ID NO: 16 is I, and the amino acid in the capsid protein corresponding to amino acid 626 of SEQ ID NO: 16 is Y;
(iv) the amino acid in the capsid protein corresponding to amino acid 77 of SEQ ID NO: 16 is R, and the amino acid in the capsid protein corresponding to amino acid 690 of SEQ ID NO: 16 is K;
(v) the amino acid in the capsid protein corresponding to amino acid 119 of SEQ ID NO: 16 is L, and the amino acid in the capsid protein corresponding to amino acid 468 of SEQ ID NO: 16 is S;
(vi) the amino acid in the capsid protein corresponding to amino acid 626 of SEQ ID NO: 16 is G, and the amino acid in the capsid protein corresponding to amino acid 718 of SEQ ID NO: 16 is G;
(vii) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(viii) the amino acid in the capsid protein corresponding to amino acid 505 of SEQ ID NO: 16 is R, and the amino acid in the capsid protein corresponding to amino acid 687 of SEQ ID NO: 16 is R;
(ix) the amino acid in the capsid protein corresponding to amino acid 346 of SEQ ID NO: 16 is A, and the amino acid in the capsid protein corresponding to amino acid 505 of SEQ ID NO: 16 is R; or
(x) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

57.-60. (canceled)

61. The method of claim 56, wherein:
a) the recombinant AAV genome comprises from 5′ to 3′: an ApoE-HCR-hAAT promoter; a composite globin/AIAT intron; a codon optimized human phenylalanine hydroxylase coding sequence; and a bovine growth hormone polyadenylation signal, or
b) the recombinant AAV genome comprises the nucleic acid sequence of SEQ ID NO: 23;
optionally, wherein the AAV capsid is an AAV5 capsid.

62.-63. (canceled)

64. A method of treating a subject having a disease or disorder, the method comprising administering to the subject a gene therapy, wherein the subject has received a first prophylactic vaccine, and wherein the prophylactic vaccine reduces the risk of occurrence and/or the severity of a first pathogenic disease in the subject.

65. The method of claim 64, wherein:
a) the first prophylactic vaccine is a herpes zoster vaccine;
b) the subject has received a second prophylactic vaccine, and wherein the second prophylactic vaccine is an S. pneumoniae vaccine; and/or
c) the subject has received a third prophylactic vaccine, and wherein the third prophylactic vaccine is an influenza vaccine, optionally wherein the disease or disorder is phenylketonuria (PKU).

66.-68. (canceled)

69. The method of claim 65, wherein the gene therapy is mediated by a recombinant viral vector, optionally, wherein the recombinant viral vector:
a) comprises a transgene encoding phenylalanine hydroxylase (PAH), or
b) is an adeno-associated virus (AAV) vector, optionally wherein the adeno-associated virus (AAV) vector comprises:
(i) an AAV capsid comprising an AAV capsid protein; and
(ii) a recombinant AAV (rAAV) genome comprising a transgene encoding a phenylalanine hydroxylase (PAH), optionally wherein the transgene comprises the nucleotide sequence set forth in SEQ ID NO: 28; and/or wherein the rAAV genome further comprises a transcriptional regulatory element operably linked to the PAH coding sequence, wherein the transcriptional regulatory element:
(a) is capable of mediating transcription in a hepatocyte, a renal cell, or a cell in the brain, pituitary gland, adrenal gland, pancreas, urinary bladder, gallbladder, colon, small intestine, or breast;
(b) comprises a human hepatic control region 1 (HCR1) comprising the nucleotide sequence set forth in SEQ ID NO: 24;
(c) comprises a human α1-antitrypsin (hAAT) promoter comprising the nucleotide sequence set forth in SEQ ID NO: 25;
(d) comprises an SV40 intron comprising the nucleotide sequence set forth in SEQ ID NO: 26; and/or
(e) comprises the nucleotide sequence set forth in SEQ ID NO: 27.

70.-79. (canceled)

80. The method of claim 69, wherein the rAAV genome:
a) further comprises an SV40 polyadenylation sequence 3′ to the PAH coding sequence, wherein the SV40 polyadenylation sequence comprises the nucleotide sequence set forth in SEQ ID NO: 29;
b) comprises the nucleotide sequence set forth in SEQ ID NO: 32;
c) further comprises a 5′ inverted terminal repeat (5′ ITR) nucleotide sequence 5′ of the genome, and a 3′ inverted terminal repeat (3′ ITR) nucleotide sequence 3′ of the genome, optionally wherein the 5′ ITR nucleotide sequence has at least 95% sequence identity to SEQ ID NO: 30, and the 3′ ITR nucleotide sequence has at least 95% sequence identity to SEQ ID NO: 31; and/or
d) comprises the nucleotide sequence set forth in SEQ ID NO: 33.

81.-84. (canceled)

85. The method of claim 80, wherein the AAV capsid comprises:
a) a capsid protein comprising an amino acid sequence having at least 95% sequence identity with the amino acid sequence of amino acids 203-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
a capsid protein comprising an amino acid sequence having at least 95% sequence identity with the amino acid sequence of amino acids 138-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
c) a capsid protein comprising an amino acid sequence having at least 95% sequence identity with the amino acid sequence of amino acids 1-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
d) a capsid protein comprising an amino acid sequence having at least 99% sequence identity with the amino acid sequence of amino acids 203-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R;
e) a capsid protein comprising an amino acid sequence having at least 99% sequence identity with the amino acid sequence of amino acids 138-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R; and/or
f) a capsid protein comprising an amino acid sequence having at least 99% sequence identity with the amino acid sequence of amino acids 1-736 of SEQ ID NO: 16, wherein the amino acid in the capsid protein corresponding to amino acid 346 of SEQ ID NO: 16 is A, and wherein the amino acid in the capsid protein corresponding to amino acid 505 of SEQ ID NO: 16 is R.

86. (canceled)

87. The method of claim 85, wherein:
a) the AAV capsid comprises a capsid protein comprising the amino acid sequence of amino acids 203-736 of SEQ ID NO: 16, a capsid protein comprising the amino acid sequence of amino acids 138-736 of SEQ ID NO: 16, and/or a capsid protein comprising the amino acid sequence of amino acids 1-736 of SEQ ID NO: 16;
b) the amino acid sequence of the capsid protein consists of the amino acid sequence of amino acids 203-736 of SEQ ID NO: 16, the amino acid sequence of the capsid protein consists of the amino acid sequence of amino acids 138-736 of SEQ ID NO: 16, and/or the amino acid sequence of the capsid protein consists of the amino acid sequence of amino acids 1-736 of SEQ ID NO: 16, wherein the transgene comprises the nucleotide sequence set forth in SEQ ID NO: 22;
c) the transcriptional regulatory element comprises one or more of:
(i) an ApoE-HCR element, optionally comprising the nucleotide sequence set forth in SEQ ID NO: 19;
(ii) a human α1-antitrypsin (hAAT) promoter comprising the nucleotide sequence set forth in SEQ ID NO: 20;
(iii) a composite globin/AIAT intron, optionally comprising the nucleotide sequence set forth in SEQ ID NO: 18; and
(iv) the nucleotide sequence set forth in SEQ ID NO: 21; and/or
d) the rAAV genome further comprises one or more of:
(i) a bovine growth hormone polyadenylation sequence;
(ii) an AAV2 5′ inverted terminal repeat (5′ ITR) nucleotide sequence 5′ of the genome, and an AAV2 3′ inverted terminal repeat (3′ ITR) nucleotide sequence 3′ of the genome, optionally, wherein the rAAV genome comprises the nucleotide sequence set forth in SEQ ID NO: 23; and optionally wherein the AAV capsid is an AAV5 capsid.

88.-97. (canceled)


SEQUENCE LISTING (34 sequences)

SEQ ID NO: 1 - protein, 736 aa, Artificial Sequence
  adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, Artificial Sequence
  AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 106 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 15 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 2002 bp, Artificial Sequence
  Synthetic polynucleotide
tgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 19 - DNA, 321 bp, Artificial Sequence
  Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 20 - DNA, 405 bp, Artificial Sequence
  Synthetic polynucleotide
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagta

SEQ ID NO: 21 - DNA, 2734 bp, Artificial Sequence
  Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 22 - DNA, 1359 bp, Artificial Sequence
  Synthetic polynucleotide
atgagcactgctgtgctggagaaccctggcctgggcagaaagctgtctgactttggccaggagaccagctacattgaggacaactgcaaccagaatggagccatcagcctgatcttcagcctgaaggaggaggtgggagccctggccaaggtgctgagactgtttgaggagaatgatgtgaacctgacccacattgagagcagacccagcagactgaagaaggatgagtatgagttcttcacccacctggacaagagaagcctgcctgccctgaccaacatcatcaagatcctgagacacgatattggagccactgtgcacgagctgagcagagacaagaagaaggacactgtgccctggttccccagaactatccaggagctggacagatttgccaaccagatcctgagctatggagctgagctggatgctgaccaccctggcttcaaggaccctgtgtacagagccagaagaaagcagtttgctgacattgcctacaactacagacacggccagcccatccccagagtggagtacatggaggaggagaagaagacctggggcactgtgttcaagaccctgaagagcctgtacaagacccacgcctgctatgagtacaaccacatcttccccctgctggagaagtactgtggcttccacgaggacaacatcccccagctggaggatgtgagccagttcctgcagacctgcactggcttcagactgagacctgtggctggcctgctgagcagcagagacttcctggggggcctggccttcagagtgttccactgcacccagtacatcagacacggcagcaagcccatgtacacccctgagcctgatatctgccacgagctgctgggccacgtgcccctgttctctgacagaagctttgcccagttcagccaggagattggcctggccagcctgggagcccctgatgagtatattgagaagctggccactatctactggttcactgtggagtttggcctgtgcaagcagggggacagcatcaaggcctatggagctggcctgctgagcagctttggggagctgcagtactgcctgtctgagaagcccaagctgctgcccctggagctggagaagactgccatccagaactacactgtgactgagttccagcccctgtactatgtggctgagagcttcaatgatgccaaggagaaggtgagaaactttgctgccaccatccccagacccttctctgtgagatatgacccctacacccagagaattgaggtgctggacaacacccagcagctgaagatcctggctgacagcatcaactctgagattggcatcctgtgctctgccctgcagaagatcaagtaa

SEQ ID NO: 23 - DNA, 4635 bp, Artificial Sequence
  Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgacgatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtatgcctttcactgcgaggggttctggagaggcttctgagctccccatggcccaggcaggcagcaggtctggggcaggaggggggttgtggagtgggtatccgcctgctgaggtgcagggcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggggctcctgtctgttgtctcctataagcctcaccacctctcctactgcttgggcatgcatctttctccccttctatagatgaggaggttaaggtccagagaggggtggggaggaacgccggctcacattctccatcccctccagatatgaccaggaacagacctgtgccaggcctcagccttacatcaaaatgggcctccccatgcaccgtggacctctgggccctcctgtcccagtggaggacaggaagctatgaggggcactgtcacccagggctcaagctggcattcctgaataatcgctctgcaccaggccacggctaagctcagtgcgtgattaagcctcataaccctccaaggcagttactagtgtgattcccattttacagatgaggaagatggggacagagaggtgaataactggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaaggtggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagttttctgattcaagacttctggcttcagctttgtacacagagatgattcaatgtcaggttttggagtgaaatctgtttaatcccagacaaaacatttaggattacatctcagttttgtaagcaagtagctctgtgatttttagtgagttatttaatgctctttggggctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgagcactgctgtgctggagaaccctggcctgggcagaaagctgtctgactttggccaggagaccagctacattgaggacaactgcaaccagaatggagccatcagcctgatcttcagcctgaaggaggaggtgggagccctggccaaggtgctgagactgtttgaggagaatgatgtgaacctgacccacattgagagcagacccagcagactgaagaaggatgagtatgagttcttcacccacctggacaagagaagcctgcctgccctgaccaacatcatcaagatcctgagacacgatattggagccactgtgcacgagctgagcagagacaagaagaaggacactgtgccctggttccccagaactatccaggagctggacagatttgccaaccagatcctgagctatggagctgagctggatgctgaccaccctggcttcaaggaccctgtgtacagagccagaagaaagcagtttgctgacattgcctacaactacagacacggccagcccatccccagagtggagtacatggaggaggagaagaagacctggggcactgtgttcaagaccctgaagagcctgtacaagacccacgcctgctatgagtacaaccacatcttccccctgctggagaagtactgtggcttccacgaggacaacatcccccagctggaggatgtgagccagttcctgcagacctgcactggcttcagactgagacctgtggctggcctgctgagcagcagagacttcctggggggcctggccttcagagtgttccactgcacccagtacatcagacacggcagcaagcccatgtacacccctgagcctgatatctgccacgagctgctgggccacgtgcccctgttctctgacagaagctttgcccagttcagccaggagattggcctggccagcctgggagcccctgatgagtatattgagaagctggccactatctactggttcactgtggagtttggcctgtgcaagcagggggacagcatcaaggcctatggagctggcctgctgagcagctttggggagctgcagtactgcctgtctgagaagcccaagctgctgcccctggagctggagaagactgccatccagaactacactgtgactgagttccagcccctgtactatgtggctgagagcttcaatgatgccaaggagaaggtgagaaactttgctgccaccatccccagacccttctctgtgagatatgacccctacacccagagaattgaggtgctggacaacacccagcagctgaagatcctggctgacagcatcaactctgagattggcatcctgtgctctgccctgcagaagatcaagtaacctcgagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 192 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 25 - DNA, 205 bp, Artificial Sequence
  Synthetic polynucleotide
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 26 - DNA, 93 bp, Artificial Sequence
  Synthetic polynucleotide
ctctaaggtaaatataaaatttttaagtgtataatgtgttaaactactgattctaattgtttctctcttttagattccaacctttggaactga

SEQ ID NO: 27 - DNA, 398 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 28 - DNA, 1359 bp, Artificial Sequence
  Synthetic polynucleotide
atgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatga

SEQ ID NO: 29 - DNA, 133 bp, Artificial Sequence
  Synthetic polynucleotide
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 30 - DNA, 106 bp, Artificial Sequence; identical to SEQ ID NO: 14
  Synthetic polynucleotide

SEQ ID NO: 31 - DNA, 143 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 32 - DNA, 2042 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 33 - DNA, 2356 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 34 - DNA, 6020 bp, Artificial Sequence
  Synthetic polynucleotide
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa